(19) United States
(12) Patent Application Publication
(10) Pub. No.: US 2022/0054404 A1
(43) Pub. Date: Feb. 24, 2022

(54) SUBCUTANEOUS ADMINISTRATION OF NANOPARTICLES COMPRISING AN MTOR INHIBITOR AND ALBUMIN FOR TREATMENT OF DISEASES

(71) Applicant: Abraxis BioScience, LLC, Summit, NJ (US)

(72) Inventors: Neil P. DESAI, Pacific Palisades, CA (US); Shihe HOU, Millington, NJ (US)

(21) Appl. No.: 17/438,825

(22) PCT Filed: Mar. 18, 2020
(86) PCT No.: PCT/US2020/023366

§ 371 Date: Sep. 13, 2021

Related U.S. Application Data
(60) Provisional application No. 62/820,842, filed on Mar. 19, 2019.
(60) Provisional application No. 62/820,838, filed on Mar. 19, 2019.

(51) Int. Cl. A61K 9/00; A61M 5/19; A61K 47/42; A61K 47/26; A61K 31/436

(52) U.S. Cl. CPC A61K 9/0019; A61M 5/19; A61K 31/436; A61K 47/26; A61K 47/42

(57) ABSTRACT

The present invention provides compositions and devices for subcutaneously administering compositions comprising nanoparticles comprising an mTOR inhibitor and an albumin. The present application also provides methods of treating diseases by subcutaneously administering to an individual a composition comprising nanoparticles comprising an mTOR inhibitor and an albumin.

### CROSS-REFERENCE TO RELATED APPLICATIONS

This application claims priority benefit to U.S. Provisional Application No. 62/820,842, filed on Mar. 19, 2019, entitled “SUBCUTANEOUS ADMINISTRATION OF NANOPARTICLES COMPRISING AN MTOR INHIBITOR AND ALBUMIN FOR TREATMENT OF DISEASES”; and U.S. Provisional Application No. 62/820,838, filed on Mar. 19, 2019, entitled “METHODS AND COMPOSITIONS FOR TREATING PULMONARY HYPERTENSION”; each of which are incorporated herein by reference for all purposes.

### TECHNICAL FIELD

This application pertains to compositions and devices for the subcutaneous administration of nanoparticles that comprise an mTOR inhibitor and an albumin and methods thereof. The application further pertains to methods of treating an individual comprising subcutaneously administering a composition comprising nanoparticles comprising an mTOR inhibitor and an albumin.

### BACKGROUND

The mammalian target of rapamycin (mTOR) is a protein kinase known to regulate various cellular processes including cell survival, proliferation, stress, and metabolism. Many inhibitors of mTOR, including rapamycin, are effective in treating various disorders including certain cancers. Many mTOR inhibitors, such as rapamycin, are known to be poorly water soluble, thus requiring excipients such as surfactants and solvents. These excipients can cause irritation, inflammation, and reduced efficacy, particularly when administered parenterally, such as subcutaneously.

Thus, there exists a need in the art for improved formulations of nanoparticles comprising an mTOR inhibitor that are stable and/or do not cause unacceptable toxicological effects upon administration, such as upon subcutaneous administration. There is also a need to develop formulations of nanoparticles comprising mTOR inhibitors that are dried, such as lyophilized, and that can be more readily constituted and/or delivered. Finally, these exists a need in the art to reduce the risk of mishandling of dried compositions before administration.

The disclosures of all publications, patents, patent applications and published patent applications referred to herein are hereby incorporated herein by reference in their entireties.

### BRIEF SUMMARY OF THE INVENTION

The present application provides methods of treating a disease in an individual comprising subcutaneously administering to the individual a pharmaceutical composition comprising nanoparticles comprising an mTOR inhibitor and an albumin, wherein the amount of the mTOR inhibitor in the pharmaceutical composition is at a dose of about 0.1 mg/m² to about 10 mg/m² for each administration. In some embodiments, the amount of the mTOR inhibitor in the pharmaceutical composition is at a dose of about 1 mg/m² to about 10 mg/m² for each administration. In some embodiments, the amount of the mTOR inhibitor in the pharmaceutical composition is at a dose of about 5 mg/m².

In some embodiments according to any one of the methods described herein, the pharmaceutical composition further comprises a saccharide.

In some embodiments according to any one of the methods described herein, the pharmaceutical composition is administered once per week or less. In some embodiments, the pharmaceutical composition is administered once per week. In some embodiments, the pharmaceutical composition is administered twice every three weeks.

In some embodiments according to any one of the methods described herein, the disease is a cancer. In some embodiments according to any one of the methods described herein, the disease is a mitochondrial disease.

In some embodiments according to any one of the methods described herein, the individual is a human.

The present application also provides methods of delivering an effective amount of an mTOR inhibitor to a target tissue of an individual comprising subcutaneously administering a pharmaceutical composition comprising nanoparticles comprising an mTOR inhibitor and an albumin. In some embodiments, the mTOR inhibitor is rapamycin. In some embodiments, the pharmaceutical composition further comprises a saccharide. In some embodiments, the pharmaceutical composition is at a dose of about 0.1 mg/m² to about 10 mg/m². In some embodiments, the target tissue is a brain tissue of the individual.

In some embodiments according to any one of the methods described herein, the average diameter of the nanoparticles in the pharmaceutical composition is no greater than about 120 nm. In some embodiments according to any one of the methods described herein, the nanoparticles comprise the mTOR inhibitor coated with the albumin. In some embodiments according to any one of the methods described herein, the albumin is human albumin. In some embodiments according to any one of the methods described herein, the mTOR inhibitor is a limus drug. In some embodiments according to any one of the methods described herein, the mTOR inhibitor is rapamycin.

The present application also provides pharmaceutical compositions suitable for subcutaneous administration to an individual comprising: a) nanoparticles comprising an mTOR inhibitor and an albumin, and b) a saccharide. In some embodiments, the saccharide is selected from the group consisting of alginate, a starch, lactose, pullulan, hyaluronic acid, chitosan, glucose, galactose, mannose, N-acetylglucosamine, sucrose, N-acetyl-D-galactosamine, maltose, or trehalose. In some embodiments, the saccharide is sucrose. In some embodiments, the saccharide is trehalose. In some embodiments, the concentration of mTOR inhibitor in the pharmaceutical composition is at least about 5 mg/ml. In some embodiments, the concentration of mTOR inhibitor in the pharmaceutical composition is at least about 50 mg/ml. In some embodiments, the average diameter of the nanoparticles in the pharmaceutical composition is no greater than about 120 nm. In some embodiments, the nanoparticles in the pharmaceutical composition comprise the mTOR inhibitor coated with the albumin. In some embodiments, the albumin in the pharmaceutical composition is human albumin. In some embodiments, the mTOR inhibitor in the pharmaceutical composition is a limus drug. In some embodiments, the mTOR inhibitor is rapamycin.

The present application also provides devices for subcutaneously administering to an individual a pharmaceutical composition comprising nanoparticles comprising an mTOR inhibitor and an albumin, the device comprising a) a drug chamber containing a dried form of the pharmaceutical composition, and a solution chamber containing a reconstituting solution: and b) a removable divider separating the drug chamber and the solution chamber, wherein removal of the divider causes mixing of the dried pharmaceutical composition and reconstituting solution, thereby forming a reconstituted pharmaceutical composition. In some embodiments, the device is a syringe, the syringe comprising an injection needle affixed to an end of the syringe and a pusher capable of expelling the reconstituted pharmaceutical composition from the syringe. In some embodiments, the pharmaceutical composition of the device further comprises a saccharide. In some embodiments of the device, the mTOR inhibitor is a limus drug. In some embodiments of the device, the mTOR inhibitor is rapamycin.

The present application also provides kits comprising any of the devices described herein for use in treating a disease. In some embodiments, the kit further comprises instructions for using the kit to treat cancer. In some embodiments, the kit further comprises instructions for using the kit to treat a mitochondrial disease.

### BRIEF DESCRIPTION OF THE FIGURES

FIG. 1 shows rapamycin concentrations in whole blood samples taken from rats after subcutaneous (SC) or intravenous (IV) administration of a single dose of nab-rapamycin (ABI-009) between 0 and 24 hours after administration.

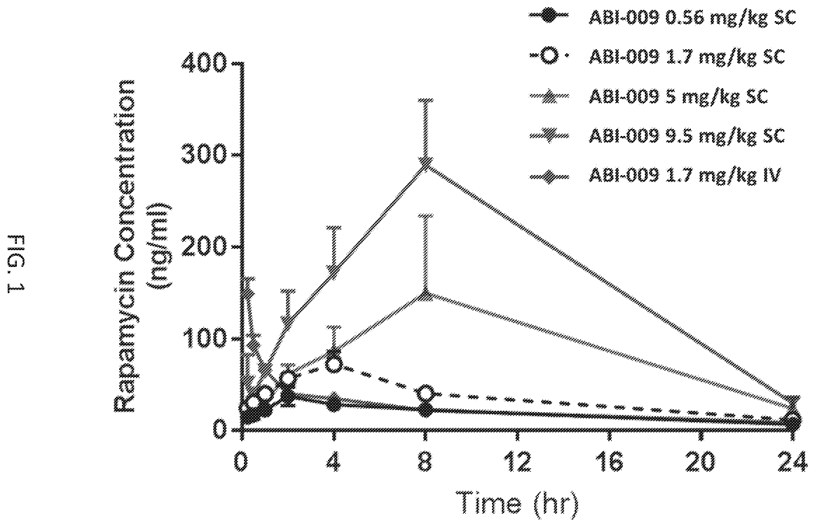

FIG. 2 shows rapamycin concentrations in whole blood samples taken from rats after subcutaneous (SC) or intravenous (IV) administration of a single dose of nab-rapamycin (ABI-009) between 0 and 168 hours after administration.

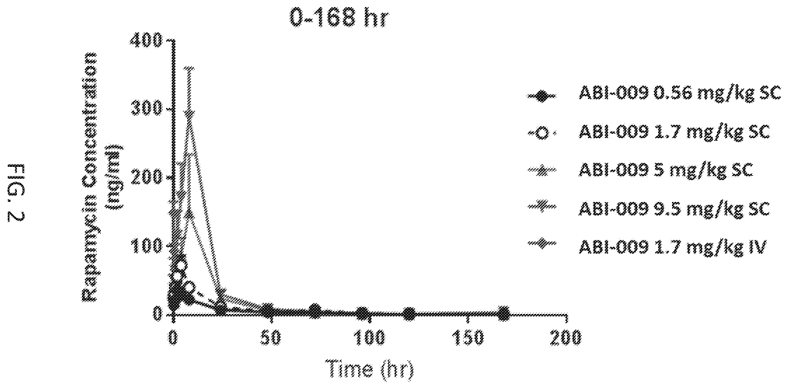

FIG. 3 shows rapamycin concentrations in whole blood samples taken from rats after subcutaneous (SC) or intravenous (IV) administration of a single dose of nab-rapamycin (ABI-009) between 0 and 24 hours after administration.

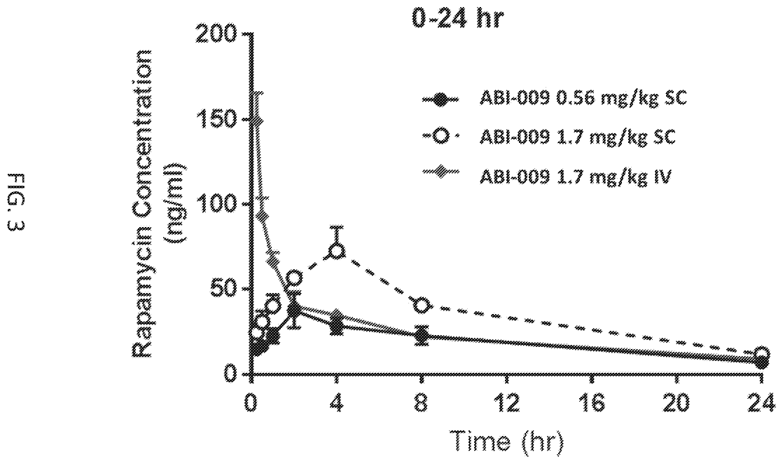

FIG. 4 shows the bioavailability of nab-rapamycin (ABI-009) after subcutaneous (subQ) or intravenous (IV) administration of a single dose in rats as indicated by the calculated area under the curve (AUC).

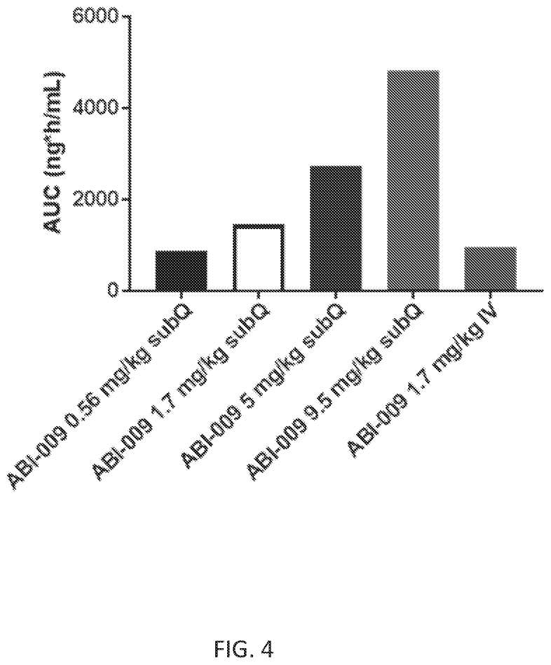

FIG. 5 shows the concentration of rapamycin in rat bone marrow (top) or brain (bottom) 24 or 168 hours after subcutaneous (subQ) or intravenous (IV) administration of a single dose of nab-rapamycin (ABI-009).

FIG. 6 shows the concentration of rapamycin in rat heart (top) or liver (bottom) 24 or 168 hours after subcutaneous (subQ) or intravenous (IV) administration of a single dose of nab-rapamycin (ABI-009).

FIG. 7 shows the concentration of rapamycin in rat lung (top) or pancreas (bottom) 24 or 168 hours after subcutaneous (subQ) or intravenous (IV) administration of a single dose of nab-rapamycin (ABI-009).

FIG. 8 shows a comparison of rapamycin concentrations over time in brain or whole blood from rats after 24, 72, and 120 post-administration of a single subcutaneous dose of nab-rapamycin (ABI-009) at a dose of 1.7 mg/kg, 9.5 mg/kg or 17 mg/kg.

FIG. 9 shows a comparison of histopathology scores assessed on skins from rats among different treatment groups.

FIG. 10 is a representative histogram image of skin from rat in Group 1 (0.9% saline). Histologic lesions are limited to an aggregate of mixed inflammatory cells (black arrow) within the subcutaneous tissues (SC). The dermis (D) and epidermis (E) are indicated.

FIG. 11 is a representative histogram image of skin from rat in Group 2 (HSA in 0.9% saline). Multifocal mixed inflammatory cell aggregates (black arrows) are visible within the subcutis (SC). The epidermis (E) and dermis (D) are unremarkable.

FIG. 12 is a representative histogram image of skin from rat in Group 3 (ABI-009, 1.7 mg/kg). Minimal mixed inflammatory cell infiltration (black arrow) is visible in the subcutaneous tissues (SC). The epidermis (E) and dermis (D) are indicated.

FIG. 13 is a representative histogram image of skin from rat in Group 4 (ABI-009, 5 mg/kg). Scattered mixed inflammatory cell infiltration (right arrow) and a site of minimal necrosis (left arrow) are present in the subcutis (SC). The epidermis (E) and dermis (D) are unremarkable.

FIG. 14 is a representative histogram image of skin from rat in Group 4 (ABI-009, 10 mg/kg). Subcutaneous (SC) mixed inflammatory cell infiltration (right arrow) and a region of necrosis (left arrow) are captured. The epidermis (E) and dermis (D) are unremarkable.

FIG. 15 shows the mean through rapamycin blood levels in rats administered with ABI-009 at 1.7 mg/kg, 5 mg/kg or 10 mg/kg.

FIG. 16 shows the tumor growth results of a human hepatocellular carcinoma mouse xenograft model after 0-15 days of treatment with saline (Group 1), ABI-009 (intravenous route; Group 2), Rapamune (oral administration; Group 3), and ABI-009 (subcutaneous route; Group 4).

FIG. 17 shows body weight changes in mice in a human hepatocellular carcinoma mouse xenograft model after 0-15 days of treatment with saline (Group 1), ABI-009 (intravenous route; Group 2), Rapamune (oral administration; Group 3), and ABI-009 (subcutaneous route; Group 4).

### DETAILED DESCRIPTION

Provided herein are methods for subcutaneously administering a composition as described herein, such as a composition comprising nanoparticles comprising an mTOR inhibitor (such as rapamycin) and an albumin. In another aspect, provided herein are methods of delivering an effective amount of an mTOR inhibitor (such as rapamycin) to a target tissue, such as brain, bone marrow, heart, liver, lung, or pancreatic tissue, by subcutaneously administering a composition comprising nanoparticles comprising an mTOR inhibitor (such as rapamycin) and an albumin. In another aspect, provided herein are methods of maintaining a blood level of an mTOR inhibitor (such as rapamycin) comprising subcutaneously administering a composition comprising nanoparticles comprising an mTOR inhibitor (such as rapamycin) and an albumin.

Also provided herein are methods of treating a disease comprising subcutaneously administering a composition comprising nanoparticles comprising an mTOR inhibitor (such as rapamycin) and an albumin. In some embodiments, the disease is a cancer. In some embodiments, an individual having a cancer is selected for treatment on the basis of having an mTOR-activating aberration. In some embodiments, the disease is a mitochondrial disorder.

Also provided herein are compositions, including pharmaceutical compositions, suitable for subcutaneous administration to an individual comprising nanoparticles comprising an mTOR inhibitor (such as rapamycin) and an albumin and methods of administering such compositions. In one aspect, the compositions may comprise one or more agents for enhancing the dissolution of dried forms of the compositions and/or enhancing the stability of the composition. The additional agent or agents may comprise a saccharide. The saccharide can be present in an amount that is effective to enhance the solubility, such as the rate of dissolution after addition of an aqueous solution to a dried form of the composition, and/or promote the stability of the compositions.

In another aspect, provided herein are devices for subcutaneously administering a pharmaceutical composition as described herein. The device comprises a drug chamber containing a dried form of the pharmaceutical composition and a solution chamber containing a reconstituting solution. The device further comprises a removable divider separating the drug chamber and the solution chamber, wherein removal or actuation of the divider causes or allows mixing of the dried pharmaceutical composition and reconstituting solution, thereby forming a reconstituted pharmaceutical composition. The device may be a syringe, which may further comprise a pusher. After reconstitution of the composition, the device is capable of, or can be adapted to be capable of, subcutaneously administering the composition to an individual. Also provided herein are methods for subcutaneously administering a composition comprising nanoparticles comprising an mTOR inhibitor and an albumin, using a device as described herein.

### Definitions

It is understood that aspects and embodiments of the invention described herein include “consisting” and/or “consisting essentially of” aspects and embodiments.

As used herein, albumin may be “associated” with an mTOR inhibitor (such as rapamycin), e.g., the composition comprises albumin-associated mTOR inhibitor. “Association” or “associated” is used herein in a general sense and refers to the albumin affecting a behavior and/or property of the mTOR inhibitor (such as rapamycin) in an aqueous composition. For example, the albumin and the mTOR inhibitor (such as rapamycin) are considered as being “associated” if the albumin makes the mTOR inhibitor (such as rapamycin) more readily suspendable in an aqueous medium as compared to a composition without the albumin. As another example, the albumin and mTOR inhibitor (such as rapamycin) are associated if the albumin stabilizes the mTOR inhibitor (such as rapamycin) in an aqueous suspension. For example, the albumin and the mTOR inhibitor can be present in a particle or a nanoparticle, which are further described herein.

General reference to a “composition” may include any of the pharmaceutical compositions described herein.

The term “effective amount” used herein refers to an amount of a compound or composition sufficient to treat a specified disorder, condition or disease such as ameliorate, palliate, lessen, and/or delay one or more of its symptoms. As is understood in the art, an “effective amount” may be in one or more doses, i.e., a single dose or multiple doses may be required to achieve the desired treatment endpoint. An effective amount may be considered in the context of administering one or more therapeutic agents, and a nanoparticle composition (e.g., a composition including rapamycin and an albumin) may be considered to be given in an effective amount if, in conjunction with one or more other agents, a desirable or beneficial result may be or is achieved.

As used herein, “nab” ® stands for nanoparticle albumin-bound, and “nab-rapamycin” is an albumin stabilized nanoparticle formulation of rapamycin. Nab-rapamycin is also known as nab-sirolimus, which has been previously described. See, for example, WO 2008/109163 A1, WO 2014/151853, WO 2008/137148 A2, and WO 2012/149451 A1, each of which is incorporated herein by reference in their entirety.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U. S. Food and Drug administration.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 109%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a.” “or.” and “the” include plural referents unless the context clearly dictates otherwise.

### Methods of Subcutaneous Administration

Provided herein are methods of subcutaneous administration of a composition, such as a pharmaceutical composition, comprising an mTOR inhibitor, such as rapamycin, and an albumin.

In some embodiments, a method is provided for delivering an effective amount of an mTOR inhibitor (such as rapamycin) to a target tissue, such as brain, bone marrow, heart, liver, lung, or pancreatic tissue, of an individual, the method comprising subcutaneously administering a composition, such as a pharmaceutical composition, comprising nanoparticles comprising mTOR inhibitor (such as rapamycin) and an albumin. In some embodiments, the individual has a tumor in the target tissue, such as the brain.

In some embodiments, a method is provided for delivering an effective amount of an mTOR inhibitor (such as rapamycin) to the brain of an individual, the method comprising subcutaneously administering a composition, such as a pharmaceutical composition, comprising nanoparticles comprising an mTOR inhibitor (such as rapamycin) and an albumin, wherein the dose of mTOR inhibitor (such as rapamycin) in the nanoparticles to deliver an effective amount of mTOR inhibitor (such as rapamycin) to the brain is any of about 0.1 mg/m² to about 10 mg/m², and values and ranges therein.

In some embodiments, the methods comprise maintaining a blood level of an mTOR inhibitor (such as rapamycin) in an individual, the method comprising subcutaneously administering a composition, such as a pharmaceutical composition, comprising nanoparticles comprising an mTOR inhibitor (such as rapamycin) and an albumin. In some embodiments, the blood level of mTOR inhibitor (such as rapamycin) is at least any of 1 ng/ml, 5 ng/ml, 10 ng/ml, 25 ng/ml, 50 ng/ml, 75 ng/ml, 100 ng/ml, 150 ng/ml, or 200 ng/ml, and values and ranges therein. In some embodiments, the individual has a tumor.

In some embodiments, provided herein is a method of treating a disease in an individual, comprising subcutaneously administering to the individual a pharmaceutical composition comprising nanoparticles comprising an mTOR inhibitor and an albumin. In some embodiments, the amount of the mTOR inhibitor in the pharmaceutical composition is at a dose of about 0.1 mg/m² to about 10 mg/m², such as about 1 mg/m² to about 10 mg/m² for each administration. In an exemplary non-limiting embodiment, the amount of the mTOR inhibitor in the pharmaceutical composition is at a dose of about 5 mg/m² for each administration.

In some embodiments, the amount of mTOR inhibitor in the composition is below the level that induces a toxicological effect (e.g., an effect above a clinically acceptable level of toxicity) or is at a level where a potential side effect can be controlled or tolerated when the mTOR inhibitor nanoparticle composition is subcutaneously administered to the individual. In some embodiments, the toxicological effect is a rash associated with subcutaneous administration of the pharmaceutical composition.

In some embodiments, the concentration of the mTOR inhibitor (such as rapamycin) in the mTOR inhibitor nanoparticle composition is between about 0.1 mg/ml and about 100 mg/ml, including for example about any of 0.1 mg/ml to about 50 mg/ml, about 0.1 mg/ml to about 50 mg/ml, about 0.1 mg/ml to about 40 mg/ml, about 0.1 mg/ml to about 10 mg/ml, or about 0.1 mg/ml to about 5 mg/ml, about 5 mg/ml to about 100 mg/ml, about 5 mg/ml to about 50 mg/ml, about 5 mg/ml to about 40 mg/ml, about 7.5 mg/ml to about 100 mg/ml, about 7.5 mg/ml to about 50 mg/ml, about 7.5 mg/ml to about 40 mg/ml, and values and ranges therein. In some embodiments, the concentration of the mTOR inhibitor (such as rapamycin) in the mTOR inhibitor nanoparticle composition is any of at least 5 mg/ml, 7.5 mg/ml, 10 mg/ml, or 20 mg/ml.

In some embodiments, the effective amount of mTOR inhibitor (such as rapamycin) in the mTOR inhibitor nanoparticle composition is in any of the following ranges: about 0.1 mg/m² to about 5 mg/m², about 5 mg/m² to about 10 mg/m², about 10 mg/m² to about 20 mg/m², about 10 to about 30 mg/m², about 10 to about 45 mg/m², about 10 to about 60 mg/m², about 20 to about 30 mg/m², about 20 to about 45 mg/m², about 20 to about 60 mg/m², about 30 to about 45 mg/m², about 30 to about 60 mg/m², or about 45 to about 60 mg/m², each inclusive. In an exemplary non-limiting embodiment, the effective amount of mTOR inhibitor (such as rapamycin) in the mTOR inhibitor nanoparticle composition is between about 0.1 mg/m² and about 10 mg/m². In another exemplary non-limiting embodiment, the effective amount of mTOR inhibitor (such as rapamycin) in the mTOR inhibitor nanoparticle composition is between about 1 mg/m² and 10 mg/m², such as 5 mg/m².

In some embodiments, the dosing frequencies for the administration of the mTOR inhibitor nanoparticle composition (such as rapamycin/albumin nanoparticle composition) include, but are not limited to, daily, every two days, every three days, every four days, every five days, every six days, weekly without break, three out of four weeks (such as on days 1, 8, and 15 of a 28-day cycle), once every three weeks, once every two weeks, or two out of three weeks. In some embodiments, the mTOR inhibitor nanoparticle composition (such as rapamycin/albumin nanoparticle composition) is administered about once every 2 weeks, once every 3 weeks, once every 4 weeks, once every 6 weeks, or once every 8 weeks. In some embodiments, the mTOR inhibitor nanoparticle composition (such as rapamycin/albumin nanoparticle composition) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 week, 2 weeks, 3 weeks, 4 weeks, 1 month, 2 months, 3 months, 4 months, 5 months, 6 months, 8 months, or 12 months. In some embodiments, there is no break in the dosing schedule. In some embodiments, the interval between each administration is no more than about a week.

The administration of the mTOR inhibitor nanoparticle composition (such as rapamycin/albumin nanoparticle composition) can be extended over an extended period of time, such as from about a month up to about seven years. In some embodiments, the mTOR inhibitor nanoparticle composition (such as rapamycin/albumin nanoparticle composition) is administered over a period of at least about any of 2, 3, 4, 5, 6, 7, 8, 9, 10, 11, 12, 18, 24, 30, 36, 48, 60, 72, or 84 months.

Auxiliary and adjuvant agents in any of the described compositions may include, for example, preserving, wetting, suspending, perfuming, emulsifying, and dispensing agents. Prevention of the action of microorganisms is generally provided by various antibacterial and antifungal agents, such as parabens, chlorobutanol, phenol, sorbic acid, and the like. Isotonic agents may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as citric acid, sorbitan monolaurate, triethanolamine oleate, butylated hydroxytoluene, and the like.

Treatments according to any dosing regimen such as the exemplary dosing regimens discussed above can be repeated for multiple cycles (such as 1, 2, 3, 4, 5, 6, or more cycles, such as about 1-10 cycles, 1-7 cycles, 1-5 cycles, 1-4 cycles, 1-3 cycles). In some embodiments, the treatment according to a specific dosing regimen is repeated for at least two, three or more cycles. In some embodiments, the treatment according to a specific dosing regimen is continuously repeated (i.e., without an interval) for at least two, three or more cycles.

In some embodiments, there is an interval between two adjacent cycles. In some embodiments, the interval is at least about one, two, three or four weeks. In some embodiments, the interval is at least about one, two, three, four, five, six or more months. In some embodiments, the interval is about a time period that allows the individual to gain weight (for example, the individual has a weight of about or at least about 90%, 92%, 95%, 97% of the weight prior to the initiation of the treatment(s) after the interval).

In some embodiments, the pharmaceutical composition is administered only once.

### Device for Sub-Cutaneous Administration

The present application demonstrates that subcutaneous administration of drugs, including mTOR inhibitor/albumin nanoparticles, exhibit better tolerability and bioavailability profiles compared to intravenous administration. In some embodiments, the compositions comprising nanoparticles comprising an mTOR inhibitor and an albumin, may store in a dried form, such a lyophilized form. To prepare the dried compositions for administration requires reconstitution with an aqueous solution, such as water. An aspect of the present application provides a device which contains a stable, fixed dose of a dried composition in close proximity to a reconstituting solution. The device comprises a divider which, through limited handling, allows for predictable and reproducible reconstitution of the dried composition followed by subcutaneous administration of the composition using the device. This device improves the handling of dried compositions by, for example, reducing the handling time of the composition, reducing the opportunities for user error, and ensuring the reproducibility and consistency of the reconstitution process. These advantages are gained, for example, by removing the step of manually adding a reconstituting solution to a dried composition, and completely removing the step of loading the reconstituted composition into a syringe.

Accordingly, provided herein are devices for subcutaneous administration of compositions, such as pharmaceutical compositions comprising nanoparticles comprising an mTOR inhibitor and an albumin. The devices described herein are particularly suitable for the subcutaneous administration of dried forms of compositions by sequential reconstitution of the dried form of the composition followed by administration of the reconstituted composition. In one aspect, the device comprises a drug chamber containing a dried form of a pharmaceutical composition, such as those described herein, such as, for example, a lyophilized form of the pharmaceutical composition, and a solution chamber containing a reconstituting solution. In another aspect, the device comprises a divider separating the drug chamber and the solution chamber. Removal of the divider, or actuation of the divider, allows, and/or causes, mixing of the dried pharmaceutical composition and reconstituting solution, thereby forms a reconstituted pharmaceutical composition. The reconstituted pharmaceutical composition can be suitable for subcutaneous administration to an individual, such as a human. In some embodiments, the device is a syringe comprising the solution chamber and the drug chamber. In some embodiments, the syringe further comprises a pusher capable of expelling the reconstituted solution from the device. In some embodiments, the syringe further comprises an injection needle, such as a hypodermic needle, suitable for subcutaneous administration affixed to an end of the syringe.

Further provided herein are compositions for subcutaneous administration contained within a device, wherein the composition comprises a lyophilized form of the pharmaceutical composition, and wherein the device comprises a solution chamber containing a reconstituting solution and a divider separating the drug chamber (containing the pharmaceutical composition) and the reconstituting solution, wherein removal or actuation of the divider allows and/or causes mixing of the dried pharmaceutical composition and reconstituting solution, thereby forming the reconstituted pharmaceutical composition.

The divider of the device may in some embodiments comprise a guard which prevents unintended removal or actuation of the divider. In some embodiments, the guard is removed from the device prior to removal or actuation of the divider. In some embodiments, the guard is actuated to allow removal or actuation of the divider.

In some embodiments, the device is a syringe comprising a suitable needle for injection. In some embodiments, the device is a syringe adapted to couple with a suitable needle for injection. Depression of the pusher of the syringe causes expulsion of the reconstituted syringe through a needle.

In some embodiments, a device is provided for subcutaneously administering a pharmaceutical composition comprising nanoparticles comprising an mTOR inhibitor (such as rapamycin) and an albumin, the device comprising a drug chamber containing a dried form of the pharmaceutical composition, a solution chamber containing a reconstituting solution, a removable divider separating the drug chamber and the solution chamber, wherein removal of the divider causes mixing of the dried pharmaceutical composition and reconstituting solution, thereby forming a reconstituted pharmaceutical composition, wherein the dose of mTOR inhibitor (such as rapamycin) in the nanoparticles is any of about 0.2 mg to about 100 mg, about 0.2 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, each inclusive.

In some embodiments, a device is provided for subcutaneously administering a pharmaceutical composition comprising nanoparticles comprising an mTOR inhibitor (such as rapamycin) and an albumin, the pharmaceutical composition optionally comprising a saccharide, the device comprising a drug chamber containing a dried form of the pharmaceutical composition, a solution chamber containing a reconstituting solution, a removable divider separating the drug chamber and the solution chamber, wherein removal of the divider causes mixing of the dried pharmaceutical composition and reconstituting solution, thereby forming a reconstituted pharmaceutical composition, wherein the dose of mTOR inhibitor (such as rapamycin) in the nanoparticles is any of about 0.2 mg to about 100 mg, about 0.2 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, each inclusive. In some embodiments, the saccharide is selected from the group consisting of alginate, a starch, lactose, pullulan, hyaluronic acid, chitosan, glucose, galactose, mannose, N-acetylglucosamine, sucrose, N-acetyl-D-galactosamine, maltose, or trehalose.

Also provided herein are methods of subcutaneous administration of a composition described herein using a device described herein. In a non-limiting exemplary embodiment, the composition comprises a dried form of a pharmaceutical composition comprising nanoparticles comprising rapamycin and an albumin. In a non-limiting exemplary embodiment, the method of subcutaneous administration comprises selecting an individual for subcutaneous administration of the pharmaceutical composition, removing or actuating the divider, waiting for an indicated period of time for the dried composition and reconstituting solution to form a reconstituted pharmaceutical composition, inserting the needle at an appropriate angle into the individual, depressing the pusher with an appropriate force, and removing the needle from the individual.

### Diseases to be Treated

The compositions, methods, and devices described herein may be useful for treating a disease or diseases in an individual, such as a human. In some embodiments, the disease or diseases are one or more of pulmonary hypertension, a central nervous system disorder, a mitochondrial disorder, or a cancer.

### I. Pulmonary Hypertension

Pulmonary hypertension (PH) is a syndrome characterized by increased pulmonary artery pressure. PH is defined hemodynamically as a systolic pulmonary artery pressure greater than 30 mm Hg or evaluation of mean pulmonary artery pressure greater than 25 mm Hg. See Zaiman et al., Am. J. Respir. Cell Mol. Biol. 33:425-31 (2005).

In some embodiments according to any one of the methods described herein, the disease or diseases to be treated comprise pulmonary hypertension. In some embodiments, the pulmonary hypertension is any of pulmonary arterial hypertension (PAH), idiopathic pulmonary arterial hypertension (IPAH), heritable pulmonary arterial hypertension (HPAH), drug and toxin induced PAH, PAH associated with connective tissue disease, and PAH associated with congenital heart defects.

In some embodiments, the pulmonary hypertension is severe pulmonary arterial hypertension. In some embodiments, the pulmonary hypertension is World Health Organization [WHO] Function Class II, III, or IV pulmonary arterial hypertension. In some embodiments, the pulmonary hypertension is WHO Function Class II pulmonary arterial hypertension. In some embodiments, the pulmonary hypertension is WHO Function Class III pulmonary arterial hypertension. In some embodiments, the pulmonary hypertension is WHO Function Class IV pulmonary arterial hypertension.

### Central Nervous System Disorders

Central nervous system diseases, also known as central nervous system disorders, are a spectrum of neurological disorders that affect the structure or function of the brain or spinal cord, which collectively form the central nervous system (CNS).

In some embodiments, the CNS disorder is a glioma. In some embodiments, the CNS disorder is a glioblastoma. In some embodiments, the CNS disorder is epilepsy. In some embodiments, the CNS disorder is cortical dysplasia (e.g., focal cortical dysplasia). In some embodiments, the CNS disorder is selected from the group consisting of tuberous sclerosis complex, brain tumor, Fragile X syndrome, Down syndrome, Rett syndrome, Alzheimer's disease, Parkinson's disease, and Huntington's disease.

In some embodiments, the CNS disorder is epilepsy. In some embodiments, the individual has undergone an epilepsy surgery. In some embodiments, the individual has at least 5 seizures in 30 days post epilepsy surgery or does not have a week of seizure freedom following epilepsy surgery. In some embodiments, the method further comprises administering to the individual an effective amount of an anti-epilepsy agent.

In some embodiments, the CNS disorder is glioblastoma. In some embodiments, the glioblastoma is recurrent glioblastoma. In some embodiments, the glioblastoma is newly diagnosed glioblastoma. In some embodiments, the individual has undergone surgical resection of newly diagnosed glioblastoma prior to the initiation of the nanoparticle administration.

### Mitochondrial Disorders

The mitochondrion is an organelle present in most eukaryotic cells. In addition to generating ATP, mitochondria are also involved in other cellular functions, such as cellular homeostasis, signaling pathways, and steroid synthesis.

Individuals having a mitochondrial-associated disorder (i.e., a mitochondrial disorder) can be treated with the methods described herein including, but not limited to, individuals having an ataxia, a kidney disorder, a liver disorder, a metabolic disorder, a myopathy, a neuropathy, a myelopathy, an encephalopathy, an oxidative phosphorylation disorder, an aging disorder, an autism spectrum disorder, a chronic inflammatory disorder, diabetes mellitus, and a fatty acid oxidation disorder. In some embodiments, the individual having a mitochondrial-associated disorder has a mitochondrial DNA mutation-associated disorder. In some embodiments, the individual having a mitochondrial-associated disorder has an X chromosome mutation-associated disorder. In some embodiments, the individual having a mitochondrial-associated disorder has a nuclear DNA mutation-associated disorder. In some embodiments, the individual having a mitochondrial-associated disorder has Leigh syndrome, such as maternally inherited Leigh syndrome. In some embodiments, Leigh syndrome is infantile onset Leigh syndrome, juvenile onset Leigh syndrome, or adult onset Leigh syndrome. In some embodiments, the individual having a mitochondrial-associated disorder has MELAS syndrome. In some embodiments, the individual having a mitochondrial-associated disorder has NARP syndrome.

Individuals having a metabolic disorder can be treated with the methods described herein including, but not limited to, disorders associated with cellular glucose consumption (e.g., abnormally high cellular glucose consumption in one or more tissues), disorders associated with insulin resistance, hypoglycemia, hyperinsulinemic hypoglycemia, diabetes mellitus type 1, diabetes mellitus type 2, and metabolic syndrome.

The methods described herein can be used for any one or more of the following purposes: alleviating one or more symptoms in an individual having a mitochondrial-associated disorder, reducing one or more symptoms in an individual having a mitochondrial-associated disorder, preventing one or more symptoms in an individual having a mitochondrial-associated disorder, treating one or more symptoms in an individual having a mitochondrial-associated disorder, ameliorating one or more symptoms in an individual having a mitochondrial-associated disorder, and delaying onset of one or more symptoms in an individual having a mitochondrial-associated disorder.

As used herein, the terms “mitochondrial-associated disorder” and “mitochondrial disorder” refer to any disease or disorder caused by dysfunction of a mitochondrion. Mitochondrial-associated disorders can cause a complex variety of symptoms. Symptoms of mitochondrial-associated disorders include, for example, muscle weakness, muscle cramps, seizures, food reflux, learning disabilities, deafness, short stature, paralysis of eye muscles, diabetes, cardiac problems, and stroke-like episodes. Symptoms of mitochondrial-associated disorders can range in severity from life-threatening to almost unnoticeable.

An individual having a mitochondrial-associated disorder can be classified in one or more subsets of mitochondrial-associated disorders based on genotype, phenotypic presentation, and/or one or more symptoms. In some embodiments, the individual having a mitochondrial-associated disorder has one or more of the following: an ataxia, a kidney disorder, a liver disorder, a metabolic disorder, a myopathy, a neuropathy, a myelopathy, an encephalopathy, an oxidative phosphorylation disorder, an aging disorder, an autism spectrum disorder, a chronic inflammatory disorder, or a fatty acid oxidation disorder. In some embodiments, the individual having a mitochondrial-associated disorder has one or more of the following: an ataxia, a kidney disorder, a liver disorder, a metabolic disorder, a myopathy, a neuropathy, a myelopathy, an encephalopathy, or an oxidative phosphorylation disorder. In some embodiments, the individual having a mitochondrial-associated disorder has one or more of the following: an aging disorder, an autism spectrum disorder, a chronic inflammatory disorder, diabetes mellitus, or a fatty acid oxidation disorder. In some embodiments, the individual having a mitochondrial-associated disorder has at least an ataxia. In some embodiments, the individual having a mitochondrial-associated disorder has at least a myelopathy and an encephalopathy. In some embodiments, the individual having a mitochondrial-associated disorder has at least a neuropathy, a myelopathy, and an encephalopathy. In some embodiments, the individual having a mitochondrial-associated disorder has at least a myopathy and a neuropathy.

### Methods of Treating Cancer

The methods described herein may be used to treat an individual having cancer with an mTOR-activating aberration at one or more genes (such as TSC1, TSC2, RPS6, PTEN, TP53, RB1, ATRX, or FAT1). In some embodiments, there is a method of treating cancer in an individual having an mTOR-activating aberration at TSC2. An individual having cancer may be selected for treatment by the methods described herein on the basis of having an mTOR-activating aberration at one or more genes (such as TSC1, TSC2, RPS6, PTEN, TP53, RB1, ATRX, or FAT1). In some embodiments, the individual is selected for treatment on the basis of having an mTOR-activating aberration at TSC2.

In some embodiments, a cancer can be treated by the methods described herein (e.g., an advanced and/or malignant cancer. e.g., PEComa, e.g., an advanced and/or malignant cancer, e.g., locally advanced inoperable cancer, e.g., a solid tumor) in an individual comprising subcutaneously administering to the individual an effective amount of a composition comprising nanoparticles comprising an mTOR inhibitor and a carrier protein, wherein the individual is selected for treatment on the basis of having an mTOR-activating aberration at TSC2. In some embodiments, a cancer can be treated by the methods described herein (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual an effective amount of a composition comprising nanoparticles comprising an mTOR inhibitor and a carrier protein, wherein the individual has an mTOR-activating aberration at TSC2. In some embodiments, the mTOR-activation aberration at TSC2 comprises a mutation in TSC2. In some embodiments, the mutation is selected from the group consisting of splice site mutation, nonsense mutation, frameshift mutation, and missense mutation. In some embodiments, the mTOR-activation aberration at TSC2 comprises a single-nucleotide variant (SNV). In some embodiments, the SNV comprises a mutation selected from the group consisting of C1503T, C2743G, C5383T, C3755G, G760T, C3442T, G880A, T707C, A4949G, or a deletion of any one or more of the amino acids at the position of 1405-1409, 1960-1970, 4999, 5002, 3521, 5208, 5238-5255. In some embodiments, the mTOR-activation aberration at TSC2 comprises a copy number variation of TSC2. In some embodiments, the mTOR-activation aberration at TSC2 is a loss of function mutation. In some embodiments, the mTOR-activation aberration in TSC2 comprises an aberrant expression level of TSC2. In some embodiments, the mTOR-activation aberration in TSC2 comprises an aberrant activity level of a protein encoded by TSC2. In some embodiments, the mTOR-activation aberration in TSC2 comprises a loss of heterozygosity of TSC2. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 0.1 mg/m² to about 100 mg/m² (e.g., about 0.1 mg/m² to about 10 mg/m², about 10 mg/m² to about 50 mg/m², about 50 mg/m² to about 100 mg/m², about 75 mg/m² to about 100 mg/m²). In some embodiments, the method comprises subcutaneously administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TSC2 aberration (e.g., a TSC2 mutation), regardless of the nature of the cancer. In some embodiments, the individual does not have a TSC1 aberration (e.g., a TSC1 mutation).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual an effective amount of a composition comprising nanoparticles comprising an mTOR inhibitor and a carrier protein, wherein the individual is selected for treatment on the basis of having a TSC2 aberration (e.g., a TSC2 mutation). In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual an effective amount of a composition comprising nanoparticles comprising an mTOR inhibitor and a carrier protein, wherein the individual is selected for treatment on the basis of a) having a TSC2 aberration (e.g., a TSC2 mutation), and b) having a RPS6 aberration (e.g., aberrant phosphorylation level of the protein encoded by RPS6 (e.g., phosphorylation at residue S235, S236, S240, and/or S244). In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual an effective amount of a composition comprising nanoparticles comprising an mTOR inhibitor and a carrier protein, wherein the individual is selected for treatment on the basis of a) having a TSC2 aberration (e.g., a TSC2 mutation), and b) not having a TSC1 mutation. In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual an effective amount of a composition comprising nanoparticles comprising an mTOR inhibitor and a carrier protein, wherein the individual is selected for treatment on the basis of a) having a TSC2 aberration (e.g., a TSC2 mutation), b) not having a TSC1 mutation, and c) having a RPS6 aberration (e.g., aberrant phosphorylation level of the protein encoded by RPS6 (e.g., phosphorylation at residue S235, S236, S240, and/or S244). In some embodiments, the mTOR-activation aberration at RPS6 comprises a positive status of phosphorylated S6 (pS6) (e.g., phosphorylation at residue S235, S236, S240, and/or S244). In some embodiments, the mutation is selected from the group consisting of splice site mutation, nonsense mutation, frameshift mutation, and missense mutation. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 0.1 mg/m² to about 100 mg/m² (e.g., about 0.1 mg/m² to about 10 mg/m², about 10 mg/m² to about 50 mg/m², about 50 mg/m² to about 100 mg/m², about 75 mg/m² to about 100 mg/m²). In some embodiments, the method comprises subcutaneously administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TSC2 aberration and a RPS6 aberration, regardless of the nature of the cancer.

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual a composition comprising nanoparticles comprising rapamycin or a derivative thereof and an albumin, wherein the individual is selected for treatment on the basis of a) having a TSC2 aberration (e.g., a TSC2 mutation), and b) having an aberrant phosphorylation level of the protein encoded by RPS6 (e.g., phosphorylation at residue S235, S236, S240, and/or S244), wherein the dose of rapamycin or a derivative thereof in the composition for each administration is from about 0.1 mg/m² to about 100 mg/m² (e.g., about 0.1 mg/m² to about 10 mg/m², about 10 mg/m² to about 25 mg/m², about 25 mg/m² to about 100 mg/m², about 50 mg/m² to about 100 mg/m², about 75 mg/m² to about 100 mg/m²), and wherein the composition is subcutaneously administered weekly for about two weeks followed by a rest period of about one week.

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising subcutaneously administering to the individual a composition comprising nanoparticles comprising rapamycin or a derivative thereof and an albumin, wherein the individual is selected for treatment on the basis of a) having a TSC2 aberration (e.g., a TSC2 mutation), b) does not have a TSC1 mutation, and c) having an aberrant phosphorylation level of the protein encoded by RPS6 (e.g., phosphorylation at residue S235, S236, S240, and/or S244), wherein the dose of rapamycin or a derivative thereof in the composition for each administration is from about 10 mg/m² to about 100 mg/m² (e.g., about 25 mg/m² to about 100 mg/m², about 50 mg/m² to about 100 mg/m², about 75 mg/m² to about 100 mg/m²), and wherein the composition is subcutaneously administered weekly for about two weeks followed by a rest period of about one week.

In some embodiments, the aberrant phosphorylation level of the protein encoded by RPS6 is a positive status of phosphorylated S6 (pS6). In some embodiments, the aberrant phosphorylation level of the protein encoded by RPS6 is an increased phosphorylation of S6 in the cancer as compared to a reference tissue. In some embodiments, the reference tissue is derived from a non-cancerous tissue in the individual. In some embodiments, the reference tissue is derived from a corresponding tissue in another individual that does not have the cancer.

In some embodiments, there is provided a method of treating a population of individuals having different cancers (e.g. advanced and/or malignant cancer, e.g., locally advanced inoperable cancer, e.g., a solid tumor), comprising subcutaneously administering to the population of individuals an effective amount of a composition comprising nanoparticles comprising an mTOR inhibitor (e.g., rapamycin) and a carrier protein (e.g., albumin), wherein each of the individuals has a TSC2 aberration (e.g., TSC2 mutation). In some embodiments, the individuals do not have a TSC1 mutation.

In some embodiments, there is provided a method of selecting an individual for a treatment on the basis of having a cancer that harbors a TSC2 mutation, wherein the treatment comprises subcutaneously administering to the individual a composition comprising nanoparticles comprising rapamycin or a derivative thereof and an albumin, wherein optionally the dose of rapamycin or a derivative thereof in the composition for each administration is from about 10 mg/m² to about 100 mg/m² (e.g., about 25 mg/m² to about 100 mg/m², about 50 mg/m² to about 100 mg/m², about 75 mg/m² to about 100 mg/m²), and wherein optionally the composition is subcutaneously administered weekly for about two weeks followed by a rest period of about one week. In some embodiments, the individual does not have a TSC1 mutation.

The cancer treated by the methods complemented in the application can be any cancer that harbors one or more mTOR-activation aberration at any of the genes selected from the group consisting of TSC1, TSC2, TP53, RB), ATRX, FA T), PTEN, and RPS6. In some embodiments, the cancer harbors one or more mTOR-activation aberration at any one of genes selected from the group consisting of TSC1, TSC2, TP53, and RPS6. In some embodiments, the cancer harbor at least one mTOR-activation aberration at RPS6 and at least one mTOR-activation aberration at TSC1, TSC2, or TP53. In some embodiments, the cancer harbor at least one mTOR-activation aberration at RPS6 and at least one mTOR-activation aberration at TSC1, or TSC2.

In some embodiments, the cancer is a solid tumor. In some embodiments, the cancer is a hematologic cancer.

In some embodiments, the cancer is advanced. In some embodiments, the cancer is malignant. In some embodiments, the cancer is an inoperable locally advanced cancer.

In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma.

In some embodiments, the cancer is a PEComa. In some embodiments, the cancer is advanced PEComa. In some embodiments, the cancer is advanced and malignant PEComa. In some embodiments, the PEComa is a uterine primary PEComa. In some embodiments, the PEComa is retroperitoneal primary PEComa. In some embodiments, the PEComa is kidney primary PEComa. In some embodiments, the PEComa is lung primary PEComa. In some embodiments, the PEComa is pelvis primary PEComa.

TSC2 is also known as Tuberin, Tuberous sclerosis 2 protein, protein phosphatase 1 regulatory subunit 160, TSC4, PPP1R160, and LAM. TSC2 protein functions as part of a complex with TSC1 by negatively regulating mTORC1 signaling. In some embodiments, the nucleic acid sequence of a wildtype TSC2 gene is identified by the Genbank accession number NC_000016.10, from nucleotide 2047936 to nucleotide 2088712 on the forward strand of chromosome 16 according to the GRCh38.p2 assembly of the human genome. The wildtype TSC2 gene comprises 42 exons. A mutation of the TSC2 gene may occur in any one or any combination of the 42 exons, or in any intron or noncoding regions of the TSC2 gene.

In some embodiments, the amino acid sequence of a wildtype TSC2 protein is identified by the Genbank accession number NP_000539.2. In some embodiments, the amino acid sequence of a wildtype TSC2 protein is identified by the Genbank accession number NP_001070651.1. In some embodiments, the amino acid sequence of a wildtype TSC2 protein is identified by the Genbank accession number NP_001107854.1.

In some embodiments, the nucleic acid sequence of a cDNA encoding a wildtype TSC2 protein is identified by the Genbank accession number NM_000548.3. In some embodiments, the nucleic acid sequence of a cDNA encoding a wildtype TSC2 protein is identified by the Genbank accession number NM_001077183.1. In some embodiments, the nucleic acid sequence of a cDNA encoding a wildtype TSC2 protein is identified by the Genbank accession number NM_001114382.1.

In some embodiments, the individual is selected for treatment based on having an mTOR-activating aberration at TSC2. In some embodiments, the mTOR-activation aberration at TSC2 comprises a mutation in TSC2. In some embodiments, the mutation is selected from the group consisting of splice site mutation, nonsense mutation, frameshift mutation, and missense mutation. In some embodiments, the mTOR-activation aberration at TSC2 comprises a single-nucleotide variant (SNV). In some embodiments, the SNV comprises a mutation selected from the group consisting of C1503T, C2743G, C5383T, C3755G, G760T, C3442T, G880A, T707C, A4949G, or a deletion of any one or more of the amino acids at the position of 1405-1409, 1960-1970, 4999, 5002, 3521, 5208, 5238-5255.

In some embodiments, the mutation is a two-point mutation. In some embodiments, the mTOR-activation aberration at TSC2 is a loss of function mutation. In some embodiments, the mTOR-activation aberration at TSC2 comprises a homozygous deletion. In some embodiments, the mTOR-activation aberration at TSC2 comprises a copy number variation of TSC2. In some embodiments, the mTOR-activation aberration at TSC2 comprises an aberrant expression level of TSC2. In some embodiments, the mTOR-activation aberration at TSC2 comprises an aberrant activity level of a protein encoded by TSC2.

Ribosomal protein S6 (RPS6) is also known as S6. Ribosomes, the organelles that catalyze protein synthesis, consist of a small 40S subunit and a large 60S subunit. Together these subunits are composed of 4 RNA species and approximately 80 structurally distinct proteins. This gene encodes a cytoplasmic ribosomal protein that is a component of the 40S subunit. The protein belongs to the S6E family of ribosomal proteins. It is the major substrate of protein kinases in the ribosome, with subsets of five C-terminal serine residues phosphorylated by different protein kinases. Phosphorylation is induced by a wide range of stimuli, including growth factors, tumor-promoting agents, and mitogens. Dephosphorylation occurs at growth arrest. The protein may contribute to the control of cell growth and proliferation through the selective translation of particular classes of mRNA. As is typical for genes encoding ribosomal proteins, there are multiple processed pseudogenes of this gene dispersed through the genome.

In some embodiments, the nucleic acid sequence of a wildtype RPS6 gene is identified by the Genbank accession number NC_000009.12, from nucleotide 19375715 to nucleotide 19380236 on the forward strand of chromosome 9 according to the GRCh38.p13 assembly of the human genome. The wildtype RPS6 gene comprises 6 exons. A mutation of the RPS6 gene may occur in any one or any combination of the 6 exons, or in any intron or noncoding regions of the RPS6 gene.

In some embodiments, the amino acid sequence of a wildtype RPS6 protein is identified by the Genbank accession number NM_001010.3.

In some embodiments, the individual is selected for treatment on the basis of having an mTOR-activating aberration at RPS6. In some embodiments, the mTOR-activation aberration at RPS6 comprises an aberrant phosphorylation level of the protein encoded by RPS6 (e.g., phosphorylation at residue S235, S236, S240, and/or S244). In some embodiments, the aberrant phosphorylation level of the protein encoded by RPS6 is a positive status of phosphorylated S6 (pS6). In some embodiments, the aberrant phosphorylation level of the protein encoded by RPS6 is an increased phosphorylation of S6 in the cancer as compared to a reference tissue. In some embodiments, the reference tissue is derived from a non-cancerous tissue in the individual. In some embodiments-, the reference tissue is derived from a corresponding tissue in another individual that does not have the cancer. The status of phosphorylated S6 can be assessed via IHC staining with an antibody that binds to phosphorylated residue(s) in S6 (e.g., an antibody that detects endogenous levels of ribosomal protein S6 only when phosphorylated at Ser235 and 236). In some embodiments, the expression level of RPS6 is assessed by immunohistochemistry. In some embodiments, the mTOR-activation aberration at RPS6 comprises an aberrant expression level of RPS6.

mTOR Inhibitors

The methods described herein in some embodiments comprise subcutaneous administration of nanoparticle compositions of mTOR inhibitors. mTOR is a serine/threonine-specific protein kinase downstream of the phosphatidylinositol 3-kinase (PI3K)/Akt (protein kinase B) pathway, and a key regulator of cell survival, proliferation, stress, and metabolism, mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004). Nat. Cell Biol, 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor is an inhibitor of mTORC1. In some embodiments, the mTOR inhibitor is an inhibitor of mTORC2. In some embodiments, the mTOR inhibitor is an inhibitor of both mTORC1 and mTORC2.

In some embodiments, the mTOR inhibitor is a limus drug. Examples of limus drugs include, but are not limited to, rapamycin, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is rapamycin. Rapamycin is a macrolide antibiotic that complexes with FKBP-12 and inhibits the mTOR pathway by binding mTORC1.

In some embodiments, the mTOR inhibitor is selected from the group consisting of rapamycin (sirolimus), BEZ235 (NVP-BEZ235), everolimus (also known as RAD001, Zortress, Certican, and Afinitor), AZD8055, temsirolimus (also known as CCI-779 and Torisel). CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquilonine derivative that is an mTORC1 catalytic inhibitor (Roper J, et al. PLoS One, 2011, 6(9), e25132). Everolimus is the 40-O-(2-hydroxyethyl) derivative of rapamycin and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1complex. P1-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014. NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific and ATP-competitive inhibitor of mTOR. Deforolimus (Ridaforolimus, AP23573, MK-8669) is a selective mTOR inhibitor.

### Nanoparticle Compositions

The mTOR inhibitor nanoparticle compositions described herein comprise nanoparticles comprising (in various embodiments consisting essentially of or consisting of) an mTOR inhibitor (such as rapamycin) and an albumin (such as human serum albumin). Nanoparticles of poorly water soluble drugs (such as macrolides) have been disclosed in, for example, U.S. Pat. Nos. 5,916,596 A; 6,506,405 B1; 6,749,868 B1, 6,537,579 B1, 7,820,788 B2, and 8,911,786 B2, and also in U S 2006/0263434 A1, US 2007/0082838 A1, and W0 2008/137148 A2, each of which is incorporated herein by reference in their entirety.

Described herein are compositions, such as pharmaceutical compositions, comprising nanoparticles comprising an mTOR inhibitor and an albumin. The mTOR inhibitors are agents selected from the compounds that inhibit the mammalian target of rapamycin (mTOR). In some embodiments, the mTOR inhibitor is rapamycin (also known as sirolimus) or an analog thereof. In some embodiments, the mTOR inhibitor is a limus drug, which includes rapamycin and its analogs. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223. In some embodiments. the mTOR inhibitor is selected from the group consisting of rapamycin (sirolimus), BEZ235 (NVP-BEZ235), everolimus (also known as RAD001, Zortress, Certican, and Afinitor), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

In some embodiments, the pharmaceutical compositions further comprise an agent or agents for enhancing dissolution of dried forms of the compositions and/or enhancing the stability of the composition. In some embodiments, the additional agent or agents comprise a saccharide. The saccharide may be, but is not limited to, monosaccharides, disaccharides, polysaccharides, and derivatives or modifications thereof. The saccharide may be, for example, any of mannitol, sucrose, fructose, lactose, maltose, dextrose, or trehalose. In some embodiments, the additional agent or agents comprise glycine. The present application therefore in one aspect provides a pharmaceutical composition suitable for subcutaneous administration to an individual comprising a) nanoparticles comprising an mTOR inhibitor (such as rapamycin) and an albumin, and b) a saccharide.

In some embodiments, the saccharide is present in an amount that is effective to increase the stability of the nanoparticles in the composition as compared to a nanoparticle composition without the saccharide. In some embodiments, the saccharide is in an amount that is effective to improve filterability of the nanoparticle composition as compared to a composition without the saccharide.

In some embodiments, the saccharide is present in an amount effective to enhance the solubility of the pharmaceutical composition. In some embodiments, the enhanced solubility comprises improved rate of dissolution of a dried form of the nanoparticle composition after addition of a reconstituting solution.

In some embodiments, the saccharide is present in an amount that reduces the incidence or severity of post-administration side effects when the nanoparticle composition is administered subcutaneously. For example, in some embodiments, the side effect is rash and the composition comprises nanoparticles comprising an mTOR inhibitor and an albumin and the saccharide is present in an amount that reduces the incidence of rash after subcutaneous administration of the nanoparticle composition.

In some embodiments, the pharmaceutical composition comprises nanoparticles comprising an mTOR inhibitor and an albumin, wherein the weight ratio of the albumin to the mTOR inhibitor in the composition is about 0.01:1 to about 100:1. In some embodiments, the composition comprises nanoparticles comprising an mTOR inhibitor (such as rapamycin) and an albumin, wherein the weight ratio of the albumin to the mTOR inhibitor (such as rapamycin) in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the composition comprises nanoparticles comprising rapamycin, or a derivative thereof, and an albumin, wherein the weight ratio of the albumin to the rapamycin or derivative thereof in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the mTOR inhibitor (such as rapamycin) is coated with albumin.

In some embodiments, the particles (such as nanoparticles) described herein have an average or mean diameter of no greater than about any of 1000, 900, 800, 700, 600, 500, 400, 300, 200, 150, 120, and 100 nm. In some embodiments, the average or mean diameter of the particles is no greater than about 200 nm. In some embodiments, the average or mean diameter of the particles is between about 20 nm to about 400 nm. In some embodiments, the average or mean diameter of the particles is between about 40 nm to about 200 nm. In some embodiments, the average or mean diameter of the nanoparticles is about 100-120 nm, for example about 100 nm. In some embodiments, the average mean diameter of the particles is less than or equal to 120 nm. In some embodiments, the average mean diameter of the particles is about 100-120 nm, for example about 100 nm. In some embodiments, the particles are sterile-filterable. Methods of determining average particle sizes are known in the art, for example, dynamic light scattering (DLS) has been routinely used in determining the size of submicrometre-sized particles. International Standard ISO022412 Particle Size Analysis—Dynamic Light Scattering, International Organisation for Standardisation (ISO) 2008 and Dynamic Light Scattering Common Terms Defined, Malvern Instruments Limited, 2011. In some embodiments, the particle size is measured as the volume-weighted mean particle size (Dv50) of the nanoparticles in the composition.

The compositions described herein may be a stable aqueous suspension of the mTOR inhibitor, such as a stable aqueous suspension of the mTOR inhibitor at a concentration of any of about 0.1 to about 200 mg/ml, about 0.1 to about 150 mg/ml, about 0.1 to about 100 mg/ml, about 0.1 to about 50 mg/ml, about 0.1 to about 20 mg/ml, about 1 to about 10 mg/ml, about 2 mg/ml to about 8 mg/ml, about 4 to about 6 mg/ml, and about 5 mg/ml. In some embodiments, the concentration of the mTOR inhibitor is at least about any of 0.2 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, 50 mg/ml, 100 mg/ml, 150 mg/ml, or 200 mg/ml.

In some embodiments, the composition is a dry (such as lyophilized) composition that can be reconstituted, resuspended, or rehydrated to form generally a stable aqueous suspension of the nanoparticles comprising an mTOR inhibitor and an albumin. In some embodiments, the composition is a liquid (such as aqueous) composition obtained by reconstituting or resuspending a dry composition. In some embodiments, the composition is an intermediate liquid (such as aqueous) composition that can be dried (such as lyophilized).

In some embodiments, the nanoparticles comprising the mTOR inhibitor (such as rapamycin) are associated (e.g., coated) with an albumin (such as human albumin or human serum albumin). In some embodiments, the composition comprises an mTOR inhibitor (such as rapamycin) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as rapamycin) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as rapamycin) that is substantially free of polymeric materials (such as polymeric matrix).

In some embodiments, the composition comprises an albumin in both nanoparticle and non-nanoparticle portions of the composition, wherein at least about any one of 50%. 60%, 70%, 80%, 90%, 95%, or 99% of the albumin in the composition are in non-nanoparticle portion of the composition.

In some embodiments, the weight ratio of an albumin (such as human albumin or human serum albumin) and a mTOR inhibitor (such as rapamycin) in the mTOR inhibitor nanoparticle composition is about 18:1 or less, such as about 15:1 or less, for example about 10:1 or less. In some embodiments, the weight ratio of an albumin (such as human albumin or human serum albumin) and an mTOR inhibitor (such as rapamycin) in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of an albumin and an mTOR inhibitor (such as rapamycin) in the nanoparticle portion of the composition is about any one of 1:2, 1:3, 1:4, 1:5, 1:9, 1:10, 1:15, or less. In some embodiments, the weight ratio of the albumin (such as human albumin or human serum albumin) and the mTOR inhibitor (such as rapamycin) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

The nanoparticles described herein may be present in a dry formulation (such as lyophilized composition) or suspended in a biocompatible medium, such as a reconstituting solution. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys34), and a single tryptophan (Trp214). Intravenous use of HSA solution has been indicated for the prevention and treatment of hypovolemic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 43946 (1999). He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)).

In some embodiments, the composition described herein is substantially free (such as free) of surfactants, such as Cremophor (or polyoxyethylated castor oil, including Cremophor EL® (BASF)). In some embodiments, the mTOR inhibitor nanoparticle composition (such as rapamycin/albumin nanoparticle composition) is substantially free (such as free) of surfactants. A composition is “substantially free of Cremophor” or “substantially free of surfactant” if the amount of Cremophor or surfactant in the composition is not sufficient to cause one or more side effect(s) in an individual when the mTOR inhibitor nanoparticle composition (such as rapamycin/albumin nanoparticle composition) is administered to the individual. In some embodiments, the mTOR inhibitor nanoparticle composition (such as rapamycinalbumin nanoparticle composition) contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in the composition described herein will vary depending on other components in the composition. In some embodiments, the composition comprises an albumin in an amount that is sufficient to stabilize the mTOR inhibitor (such as rapamycin) in an aqueous suspension, for example, in the form of a stable colloidal suspension (such as a stable suspension of nanoparticles). In some embodiments, the albumin is in an amount that reduces the sedimentation rate of the mTOR inhibitor (such as rapamycin) in an aqueous medium. For particle-containing compositions, the amount of the albumin also depends on the size and density of nanoparticles of the mTOR inhibitor.

An mTOR inhibitor (such as rapamycin) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed using an optical microscope at 1000 times, at about fifteen minutes after preparation of the suspension. Stability can also be evaluated under accelerated testing conditions, such as at a temperature that is about 40° C. or higher.

In some embodiments, the albumin is present in an amount that is sufficient to stabilize the mTOR inhibitor (such as rapamycin) in an aqueous suspension at a certain concentration. For example, the concentration of the mTOR inhibitor (such as rapamycin) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as rapamycin) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the composition, in liquid form, comprises about 0.5% to about 5% (w/v) of albumin.

In some embodiments, the weight ratio of the albumin to the mTOR inhibitor (such as rapamycin) in the mTOR inhibitor nanoparticle composition is such that a sufficient amount of mTOR inhibitor binds to, or is transported by, the cell. While the weight ratio of an albumin to an mTOR inhibitor (such as rapamycin) will have to be optimized for different albumin and mTOR inhibitor combinations, generally the weight ratio of an albumin to an mTOR inhibitor (such as rapamycin) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as rapamycin) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as rapamycin) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to an individual (such as a human) without significant side effects. In some embodiments, the albumin (such as human serum albumin or human albumin) is in an amount that is effective to reduce one or more side effects of subcutaneous administration of the mTOR inhibitor (such rapamycin) to a human. The term “reducing one or more side effects” of administration, such as subcutaneous administration, of the mTOR inhibitor (such as rapamycin) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as rapamycin) can be reduced.

In some embodiments, the mTOR inhibitor nanoparticle compositions described herein comprise nanoparticles comprising an mTOR inhibitor (such as rapamycin) and an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as rapamycin) and an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as rapamycin) and an albumin (such as human albumin or human serum albumin), wherein the nanoparticles have an average diameter of no greater than about 150 nm (for example about 100 nm). In some embodiments, the average or mean diameter of the nanoparticles is about 100-120 nm, for example about 100 nm. In some embodiments, the mTOR inhibitor nanoparticle compositions described herein comprise nanoparticles comprising rapamycin and human albumin (such as human serum albumin), wherein the nanoparticles have an average diameter of no greater than about 150 nm (for example about 100 nm). In some embodiments, the average or mean diameter of the nanoparticles is about 100-120 nm, for example about 100 nm. In some embodiments, the mTOR inhibitor nanoparticle compositions described herein comprise nanoparticles comprising rapamycin and human albumin (such as human serum albumin), wherein the average or mean diameter of the nanoparticles is about 10 to about 150 nm. In some embodiments, the mTOR inhibitor nanoparticle compositions described herein comprise nanoparticles comprising rapamycin and human albumin (such as human serum albumin), wherein the average or mean diameter of the nanoparticles is about 40 to about 120 nm.

In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 200 nm. In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 150 nm. In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 150 nm (for example about 100 nm). In some embodiments, the average or mean diameter of the nanoparticles is about 100-120 nm, for example about 100 nm. In some embodiments, the mTOR inhibitor nanoparticle compositions described herein comprise nanoparticles comprising rapamycin and human albumin (such as human serum albumin), wherein the composition further comprises a saccharide, wherein the nanoparticles have an average diameter of no greater than about 150 nm (for example about 100 nm). In some embodiments, the average or mean diameter of the nanoparticles is about 100-120 nm, for example about 100 nm. In some embodiments, the mTOR inhibitor nanoparticle compositions described herein comprise nanoparticles comprising rapamycin and human albumin (such as human serum albumin), wherein the composition further comprises a saccharide, wherein the average or mean diameter of the nanoparticles is about 10 to about 150 nm. In some embodiments, the mTOR inhibitor nanoparticle compositions described herein comprise nanoparticles comprising rapamycin and human albumin (such as human serum albumin), wherein the average or mean diameter of the nanoparticles is about 40 to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) and an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) and an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin and human albumin (such as human serum albumin), wherein the nanoparticles have an average diameter of no greater than about 150 nm (for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin and human albumin (such as human serum albumin), wherein the composition further comprises a saccharide, wherein the nanoparticles have an average diameter of no greater than about 150 nm (for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) associated (e.g., coated) with an albumin (such as human albumin or human serum albumin). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of about 10 nm to about 150 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of about 40 nm to about 120 nm. In some embodiments, the mTOR inhibitor nanoparticle compositions described herein comprise nanoparticles comprising rapamycin associated (e.g., coated) with human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising rapamycin associated (e.g., coated) with human albumin (such as human serum albumin), wherein the nanoparticles have an average diameter of about 10 nm to about 150 nm. In some embodiments, the mTOR inhibitor nanoparticle compositions described herein comprise nanoparticles comprising rapamycin associated (e.g., coated) with human albumin (such as human serum albumin), wherein the nanoparticles have an average diameter of about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 200 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 150 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of about 10 nm to about 150 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of about 40 nm to about 120 nm. In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of no greater than about 150 nm (for example about 100 nm). In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of about 10 nm to about 150 nm. In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin associated (e.g., coated) with human albumin (such as human serum albumin), wherein the nanoparticles have an average diameter of no greater than about 150 nm (for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of no greater than about 150 nm (for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) stabilized by an albumin (such as human albumin or human serum albumin). In some embodiments, the mTOR inhibitor nanoparticle compositions described herein comprise nanoparticles comprising an mTOR inhibitor (such as rapamycin) stabilized by an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as rapamycin) stabilized by an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as rapamycin) stabilized by an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising rapamycin stabilized by human albumin (such as human serum albumin), wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide. In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 200 nm. In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 150 nm. In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 150 nm (for example about 100 nm). In some embodiments, the mTOR inhibitor nanoparticle compositions described herein comprise nanoparticles comprising rapamycin stabilized by human albumin (such as human serum albumin), wherein the composition further comprises a saccharide, wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) stabilized by an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin stabilized by human albumin (such as human serum albumin), wherein the nanoparticles have an average diameter of no greater than about 150 nm (for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin stabilized by human albumin (such as human serum albumin), wherein the composition further comprises a saccharide, wherein the nanoparticles have an average diameter of no greater than about 150 nm (for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle composition comprises nab-rapamycin. In some embodiments, the mTOR inhibitor nanoparticle composition is nab-rapamycin. Nab-rapamycin is a formulation of rapamycin stabilized by human albumin USP, which can be dispersed in directly injectable physiological solution. The weight ratio of human albumin and rapamycin is about 8:1 to about 9:1. When dispersed in a suitable aqueous medium such as 0.9% sodium chloride injection or 5% dextrose injection, nab-rapamycin forms a stable colloidal suspension of rapamycin. The mean particle size of the nanoparticles in the colloidal suspension is about 100 nanometers. Since HSA is freely soluble in water, nab-rapamycin can be reconstituted in a wide range of concentrations ranging from dilute (0.1 mg/ml rapamycin or a derivative thereof) to concentrated (20 mg/ml rapamycin or a derivative thereof), including for example about 2 mg/ml to about 8 mg/ml, or about 5 mg/ml.

Methods of making nanoparticle compositions are known in the art. For example, nanoparticles containing an mTOR inhibitor (such as rapamycin) and an albumin (such as human serum albumin or human albumin) can be prepared under conditions of high shear forces (e.g., sonication, high pressure homogenization, or the like). These methods are disclosed in, for example, U. S. Pat. Nos. 5,916,596; 6,506,405; 6,749,868, 6,537,579, 7,820,788, and 8,911,786, and also in U. S. Pat. Pub. Nos. 2007/0082838, 2006/0263434 and PCT Application WO08/137148.

Briefly, the mTOR inhibitor (such as rapamycin) is dissolved in an organic solvent, and the solution can be added to an albumin solution. The mixture is subjected to high pressure homogenization. The organic solvent can then be removed by evaporation. The dispersion obtained can be further lyophilized. Suitable organic solvents include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

Other Components in the mTOR Inhibitor Nanoparticle Compositions

The nanoparticles described herein can be present in a composition that includes other agents, carriers, excipients, diluents, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palrmitoyloleoylphosphatidylcholine, palmitoyllinoleovlphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. In some embodiments, the composition is suitable for administration after reconstitution.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, and/or preserving agents.

Formulations suitable for subcutaneous administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, immediately prior to use. Extemporaneous solutions and suspensions can be prepared from sterile powders, granules, and tablets of the kind previously described.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits

In some embodiments, there is provided a kit useful for various purposes, e.g., for treatment of a disease in an individual. Kits of the invention include one or more containers comprising an mTOR inhibitor nanoparticle composition (such as rapamycin/albumin nanoparticle composition) (or unit dosage form and/or article of manufacture) suitable for sub-cutaneous administration, and in some embodiments, further comprise a device for subcutaneously administering the mTOR inhibitor nanoparticle composition. In some embodiments, the kit further comprises instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of the invention are in suitable packaging. Suitable packaging includes, but is not limited to, vials, bottles, jars, flexible packaging (e.g., sealed Mylar® or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the mTOR inhibitor nanoparticle composition generally include information as to dosage, dosing schedule, and route of administration for the intended treatment. The containers may be unit doses, bulk packages (e.g., multi-dose packages) or sub-unit doses. For example, kits may be provided that contain sufficient dosages of an mTOR inhibitor nanoparticle composition (such as rapamycin/albumin nanoparticle composition) to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor nanoparticle composition (such as rapamycin/albumin nanoparticle composition) and instructions for use, packaged in quantities sufficient for storage and use in pharmacies, for example, hospital pharmacies and compounding pharmacies.

The kits may further comprise a device which contains the mTOR inhibitor nanoparticle composition. The instructions may further comprise instructions for use of the device.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1: Pharmacokinetics Study Following Subcutaneous and Intravenous Dosing of ABI-009 in Sprague Dawley (SD) Rats

Female SD rats received a single dose of nab-rapamycin (ABI-009) subcutaneously (i.e., “SC” or “subQ”) or intravenously (IV). The study design is summarized below in Table 1. No inflammation or toxicity was observed after administration at the subcutaneous injection sites at any time point compared with the saline control (vehicle).

TABLE 1
Study Design of Single Dose of ABI-009 in Rats

| | | | | | Euthanasia |
|---|---|---|---|---|---|
| | No. | Test | Route of | | time point |
| Group | rats | material | administration | Dose | (hours) |
| 1 | 3 | vehicle | SC | 0.5 ml/kg | 168 |
| 2 | 3 | ABI-009 | SC | 0.56 mg/kg | 24 |
| 3 | 4 | ABI-009 | SC | 0.56 mg/kg | 168 |
| 4 | 3 | ABI-009 | SC | 1.7 mg/kg | 24 |
| 5 | 3 | ABI-009 | SC | 1.7 mg/kg | 168 |
| 6 | 3 | ABI-009 | SC | 5 mg/kg | 24 |
| 7 | 3 | ABI-009 | SC | 5 mg/kg | 168 |
| 8 | 3 | ABI-009 | SC | 9.5 mg/kg | 24 |
| 9 | 3 | ABI-009 | SC | 9.5 mg/kg | 168 |
| 10 | 3 | ABI-009 | IV | 1.7 mg/kg | 24 |
| 11 | 3 | ABI-009 | IV | 1.7 mg/kg | 168 |

After subcutaneous or intravenous injection of ABI-009, rapamycin concentrations in the whole blood were measured at different time points. The results of the whole blood collections are summarized in Tables 2 and 3 below.

TABLE 2
Rapamycin Concentration after ABI-009 Administration

| Time | ABI-009 0.56 mg/kg SC | | | ABI-009 1.7 mg/kg SC | | | ABI-009 5 mg/kg SC | | |
|---|---|---|---|---|---|---|---|---|---|
| (hr) | Average | SD | N | Average | SD | N | Average | SD | N |
| 0.25 | 14.70 | 3.66 | 3 | 24.63 | 4.74 | 3 | 21.40 | 5.39 | 3 |
| 0.5 | 16.77 | 3.66 | 3 | 30.93 | 6.37 | 3 | 19.20 | 6.92 | 3 |
| 1 | 22.53 | 4.27 | 3 | 40.23 | 6.55 | 3 | 30.17 | 5.91 | 3 |
| 2 | 37.40 | 10.02 | 3 | 56.67 | 1.62 | 3 | 61.73 | 9.81 | 3 |
| 4 | 28.37 | 4.58 | 3 | 72.60 | 14.10 | 3 | 86.60 | 26.54 | 3 |
| 8 | 22.70 | 5.2.2 | 3 | 40.57 | 3.56 | 3 | 149.70 | 84.47 | 3 |
| 24 | 6.95 | 1.29 | 3 | 11.80 | 1.80 | 3 | 24.17 | 11.65 | 3 |
| 48 | 4.13 | 1.10 | 3 | 5.75 | 0.80 | 3 | 6.87 | 2.04 | 3 |
| 72 | 4.57 | 3.51 | 3 | 7.32 | 5.96 | 3 | 3.59 | 0.27 | 3 |
| 96 | 1.89 | 0.52 | 3 | 2.37 | 0.80 | 3 | 1.80 | 0.54 | 3 |
| 120 | 1.40 | 0.44 | 3 | 1.75 | 0.60 | 3 | 1.48 | 0.29 | 3 |
| 168 | 1.01 | 0.28 | 3 | 1.18 | 0.19 | 3 | 0.90 | 0.39 | 3 |

TABLE 3
Rapamycin Concentration after ABI-009 Administration

| Time | ABI-009 9.5 mg/kg SC | | | ABI-009 1.7 mg/kg IV | | |
|---|---|---|---|---|---|---|
| (hr) | Average | SD | N | Average | SD | N |
| 0.25 | 51.70 | 31.20 | 3 | 149.00 | 16.64 | 3 |
| 0.5 | 37.83 | 8.17 | 3 | 93.00 | 10.75 | 3 |
| 1 | 64.93 | 7.43 | 3 | 66.30 | 5.48 | 3 |
| 2 | 116.27 | 36.19 | 3 | 40.07 | 8.59 | 3 |
| 4 | 171.67 | 49.57 | 3 | 34.80 | 0.85 | 3 |
| 8 | 289.33 | 70.88 | 3 | 22.13 | 3.86 | 3 |
| 24 | 30.03 | 4.82 | 3 | 8.85 | 1.46 | 3 |
| 48 | 8.93 | 1.20 | 3 | 4.66 | 1.53 | 3 |
| 72 | 5.09 | 2.08 | 3 | 2.95 | 0.85 | 3 |
| 96 | 2.58 | 0.84 | 3 | 1.78 | 0.42 | 3 |
| 120 | 1.76 | 0.44 | 3 | 1.39 | 0.36 | 3 |
| 168 | 4.09 | 5.06 | 3 | 0.87 | 0.30 | 3 |

Surprisingly, as summarized in Table 4, below, subcutaneous administration enhanced bioavailability as indicated by total area under the curve (AUC) compared with intravenous administration. Subcutaneous administration of only 0.56 mg/kg ABI-009 produced similar drug exposure at ⅓rd the dose of IV ABI-009 (1.7 mg/kg). Further, subcutaneous administration reduced the maximum concentration achieved (Cmax) and delayed the time to reach the maximum concentration (Cmax time). Rapamycin peak levels and AUC in blood increased with higher subcutaneous ABI-009 doses.

TABLE 4
Pharmacokinetics of ABI-009 Administration in Rats

| Route | SC | SC | SC | SC | IV |
|---|---|---|---|---|---|
| Dose (mg/kg | 0.56 | 1.7 | 5 | 9.5 | 1.7 |
| Cmax (ng/mL) | 37.40 | 72.60 | 149.70 | 289.33 | 149.00 |
| Cmax Time | 2 | 4 | 8 | 8 | 0.25 |
| (h) | | | | | |
| AUC | 860.8 | 1451 | 2734 | 4813 | 962.6 |
| (ng * h/mL) | | | | | |

### Example 2: Biodistribution of ABI-009 after Administration in Rats

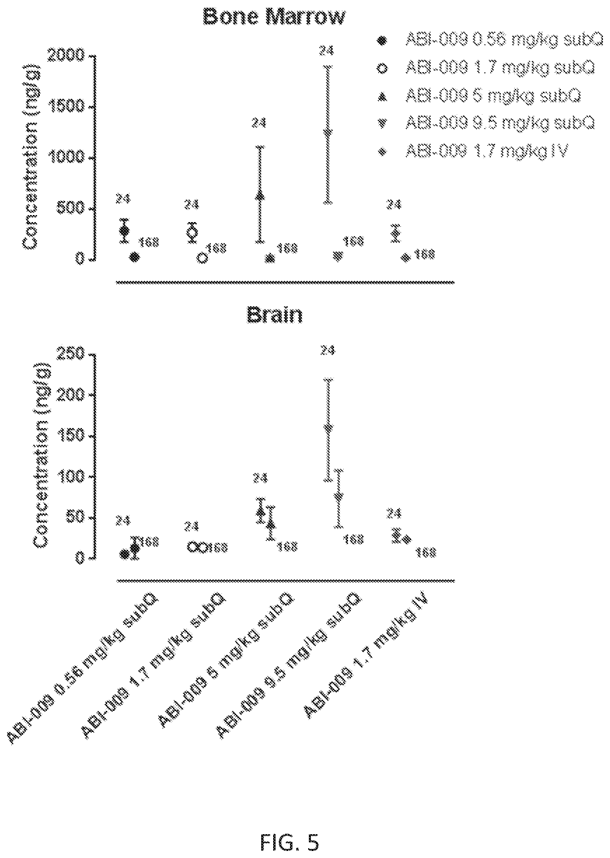

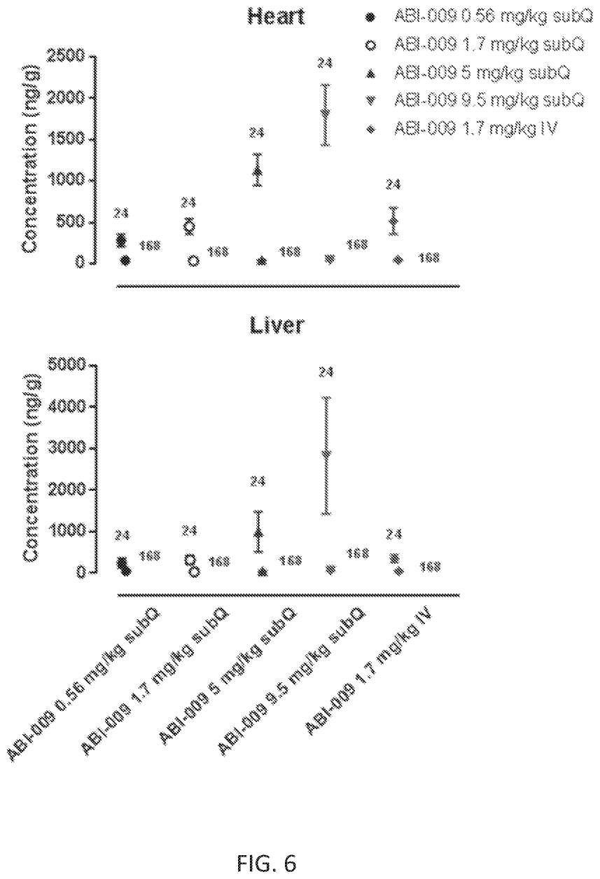

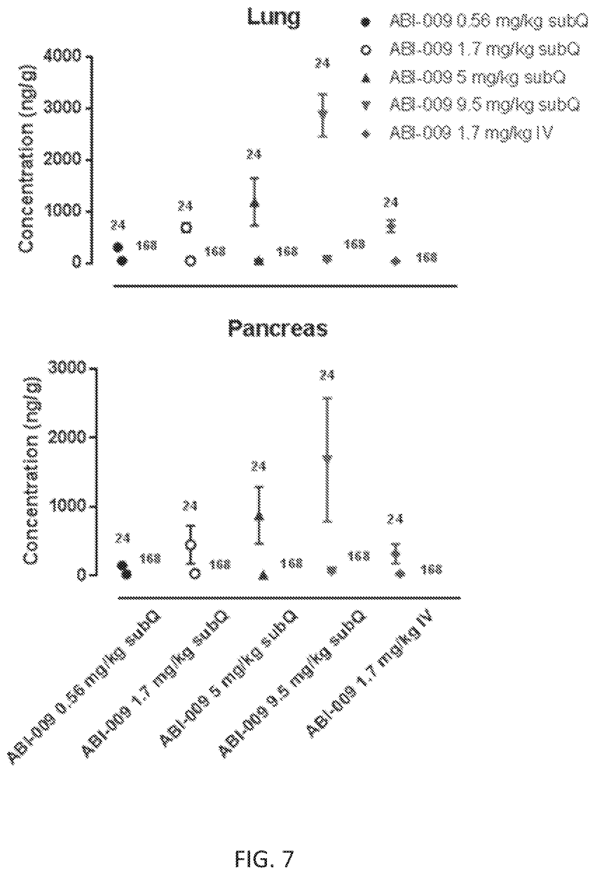

Tissues were harvested from the rats described above in Example 1 at either 24 hours or 168 hours (see Table 1 for study design) post-administration by subcutaneous (subQ) or intravenous (IV) route of ABI-009. The concentration of rapamycin in particular rat tissues 24 or 168 hours post-administration is indicated in FIG. 5 (bone marrow and brain), FIG. 6 (heart and lung), and FIG. 7 (lung and pancreas).

The subcutaneous route of administration resulted in significant distribution to all organs tested, including bone marrow, brain, heart, liver, lung, and pancreas. The pattern of organ distribution was similar between subcutaneous and intravenous but subcutaneous administration at 0.56 mg/kg dose was able to produce similar tissue concentrations as intravenous administration at 1.7 mg/kg dose. There was a significant drop in rapamycin concentration between 24 and 168 hours in well-perfused organs including the heart, liver, lung, and pancreas. However, the brain concentration was relatively stable between 24 and 168 hours.

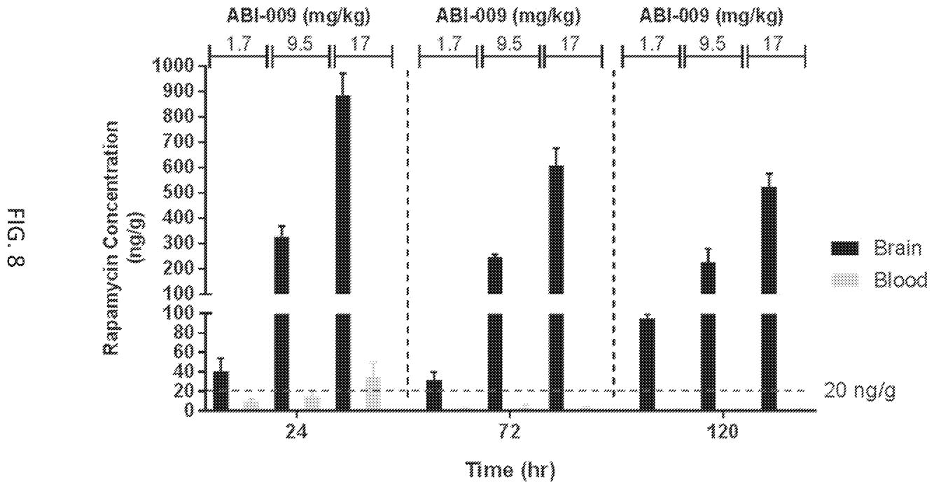

To further clarify the difference between brain and blood distribution of rapamycin, a further experiment was conducted with rats. Rats were subcutaneously administered a single dose of nab-rapamycin (ABI-009) at a dose of 1.7 mg/kg, 9.5 mg/kg, or 17 mg/kg. Rats were sacrificed at 24, 72, and 120 hours and whole blood and brain tissue were collected. Rapamycin concentrations were measured at each time points for each sample. As indicated in FIG. 5, a dose-dependent increase in brain rapamycin levels was observed. Surprisingly, while blood levels of rapamycin rapidly approached baseline, even at the high 17 mg/kg dose, brain rapamycin levels were well-maintained over the entire 120 hours, even at the lowest dose. See also FIG. 8.

### Example 3: Nab-Rapamycin Nanoparticle Formulations Containing a Sugar

Formulations of nab-rapamycin (ABI-009) will be prepared with and without saccharides, including formulations of sucrose and formulations of trehalose. The formulations will be lyophilized and then reconstituted with water at various concentrations from 1 mg/ml to 40 mg/ml rapamycin. The formulations will then be lyophilized again and incubated at 40° C. for 15 days.

After incubation, the formulations will be reconstituted with water and concurrently or subsequently assayed for albumin oligomers and polymers and reconstitution time.

Formulations that exhibit reduced albumin oligomers and polymers, and/or rapid reconstitution, will be selected as enhanced formulations for subcutaneous administration.

### Example 4: Toxicology Study Following Repeated Subcutaneous Dosing of ABI-009 in SD Rats

The objectives of the study were to assess the overall safety and local toxicity at injection sites following repeated ABI-009 SC injections in SD rats. The signs of clinical distress were observed to determine toxicity. Skin samples from the injection sites were analyzed for signs of inflammation and necrosis by histopathology.

Fifteen female Sprague Dawley (SD) rats weighing 160-180 g were used in the study. ABI-009 was dissolved in saline to prepare a stock solution (10 mg/ml), then further diluted in HSA 0.9% saline solution to prepare subcutaneous (volume: 1.0 ml/kg).

A. Study Design

Rats were divided into 5 groups of 3 animals each. Rats were weighed and dosed subcutaneously, as specified in Table 5, every 4 days for 4 weeks (7 injections).

TABLE 5
Treatment Groups

| | Number of | | | | | |
|---|---|---|---|---|---|---|
| Group | Rats | Test articles | ROA | Dose | Dose volume | Schedule |
| 1 | 3 | 0.9% Saline | SC | — | 1.0 ml/kg | Once |
| 2 | 3 | HSA in 0.9% Saline | SC | 90 mg HSA/kg | | every 4 |
| 3 | 3 | ABI-009 | SC | 1.7 mg/kg | | days for |
| 4 | 3 | ABI-009 | SC | 5 mg/kg | | 4 weeks |
| 5 | 3 | ABI-009 | SC | 10 mg/kg | | |

C = subcutaneous injection

Animals were examined daily for clinical signs of overall toxicity and the local injection sites examined for reactions to subcutaneous injection.

Whole blood samples were collected prior to each injection for animals receiving ABI-009 (Groups 3, 4, and 5) and analyzed for trough rapamycin levels.

All animals were euthanized after 4 weeks and skin samples from local injection sites were examined by histopathology for signs of local toxicity.

B. Experiment Procedures

1. Dosing Solution Preparation

Vehicle controls consist of 0.9% saline solution and HSA in 0.9% saline solution. Final concentration of HSA solution is 90 mg/ml, based on the albumin:rapamycin ratio of 9:1 of the test article ABI-009 (manufacture lot #C345-001, Fisher lot #51394.2). Each vial of ABI-009 (C345-001) contains 97.4 mg rapamycin and 874 mg human albumin. HSA saline solution is diluted from 20% Grifols albumin stock solution (200 mg/ml).

For ABI-009 dosing solutions, first make a stock ABI-009 solution of 10 mg/ml, then dilute to desired concentrations for dosing solution using HSA-saline solution. A vial of 100 mg of ABI-009 was dissolved in 10 ml of 0.9% saline to prepare a solution of 10 mg/ml.

ABI-009 solution of 5 mg/ml was prepared by diluting 0.6 ml of stock solution (10 mg/ml) with 0.6 ml of HSA-0.9% saline to prepare a solution of 5.0 mg/ml for group 4. ABI-009 solution of 1.7 mg/ml was prepared by diluting 0.3 ml of ABI-009 solution from group 4 (5.0 mg/ml) with 0.6 ml of HSA-0.9% saline to prepare a solution of 1.7 mg/ml for group 3.

2. Dosing

The rats were anesthetized, weighed, and administered with ABI-009 solutions, HSA solution and saline according to Table 6 by subcutaneous (SC) injection every 4 days for 4 weeks (7 injections).

TABLE 6
Dosing Volume

| | | | | Dosing | Dose |
|---|---|---|---|---|---|
| | | | Dose | Sol | Volume |
| Group | Test articles | ROA | (mg/kg) | (mg/ml) | (ml/kg) |
| 1 | 0.9% Saline | SC | 0 | 0 | 1.0 |
| 2 | HSA in | SC | 0 (90 | 0 (90 | 1.0 |
| | 0.9% Saline | | mg HSA) | mg HSA) | |
| 3 | ABI-009 | SC | 1.7 | 1.7 | 1.0 |
| 4 | ABI-009 | SC | 5 | 5 | 1.0 |
| 5 | ABI-009 | SC | 10 | 10 | 1.0 |

Rats were examined once daily for clinical signs of overall toxicity and the local injection sites for reactions to subcutaneous injection. The signs of clinical distress were observed to determine toxicity. Piloerection, weight loss, lethargy, discharges, neurological symptoms, morbidity, redness and inflammation of injection site, and any other signs considered abnormal for animal behavior. Pictures of the injection site for all rats were taken before and after the SC injection.

3. Sample Collection and Analysis

For rats treated with ABI-009 (Groups 3, 4, and 5), rats were anesthetized and bled for samples into pre-chilled K2EDTA tubes before each administration (except 1st dose). Whole blood was collected, stored in labeled Eppendorf tubes at −80° C., and analyzed for trough rapamycin levels.

All animals were euthanized at the final euthanasia points of Day 29 (96 hrs post week 4 Day 25 ABI-009 administrations). At the final euthanasia time point, whole blood samples were collected for analysis of trough rapamycin level. The brain, lung, liver, heart, pancreas, and bone marrow were collected, flushed with saline to remove the blood, divided into 2 portions, and flash frozen in individually labeled tubes, and stored at −80° C. The frozen blood samples from ABI-009 treated groups (Groups 3, 4, and 5) are shipped on dry ice to BASi. Trough rapamycin blood levels were analyzed by BASi by LC/MS/MS method.

At the final euthanasia time point, skin and lower dermal layer at region of SC administration were excised for histological analysis by H&E staining for signs of inflammation by histopathology. Fifteen formalin-fixed rat skin samples were subject to histopathologic measurement and processed routinely. One slide from each block was sectioned and stained with hematoxylin and eosin (H&E). Slides were evaluated by a board-certified veterinary pathologist using light microscopy. Histologic lesions were graded for severity 0-5 (0=not present/normal, 1=minimal, 2=mild, 3=moderate, 4=marked, 5=severe). Mean scores of different groups were analyzed by t-test.

C. Results

1. Systemic Toxicity

The signs of clinical distress were observed daily to determine toxicity. Piloerection, weight loss, lethargy, discharges, neurological symptoms, morbidity, redness and inflammation of injection site, and any other signs considered abnormal for animal behavior. Rats were normal post dosing of saline. HSA, and ABI-009 at current dose regimen (17-10 mg/kg, 7 doses), with no signs of clinic stress observed during the study.

There was no body weight loss (<20%), and all treatment groups gained weight during the study (Table 7). The results showed that rats tolerated subcutaneous injection of ABI-009 over a dose range of 1.7-10.0 mg/kg.

TABLE 7
Effect of Treatment on the Body Weight of Rats

| | | Body weight (g) | | | | | | |
|---|---|---|---|---|---|---|---|---|
| Groups | Mouse # | Day 1 | Day 5 | Day 9 | Day 13 | Day 17 | Day 21 | Day 25 |
| Group 1 | 1 | 181 | 187 | 195 | 202 | 207 | 210 | 213 |
| 0.9% saline | 2 | 200 | 196 | 206 | 210 | 214 | 218 | 228 |
| 1 ml/kgg | 3 | 187 | 191 | 193 | 201 | 204 | 209 | 219 |
| | average | 189 | 191 | 198 | 204 | 208 | 212 | 220 |
| | SD | 9.71 | 4.51 | 7.00 | 4.93 | 5.13 | 4.93 | 7.55 |
| Group 2 | 4 | 182 | 188 | 196 | 201 | 210 | 212 | 222 |
| HSA in 0.9% saline | 5 | 197 | 200 | 208 | 214 | 221 | 226 | 239 |
| 1 ml/kg | 6 | 173 | 180 | 188 | 199 | 207 | 211 | 216 |
| | average | 184 | 189 | 197 | 205 | 213 | 216 | 226 |
| | SD | 12.12 | 10.07 | 10.07 | 8.14 | 7.37 | 8.39 | 11.93 |
| Group 3 | 7 | 191 | 189 | 192 | 199 | 207 | 206 | 215 |
| ABI-009 | 8 | 186 | 189 | 186 | 193 | 199 | 200 | 209 |
| 1.7 mg/kg | 9 | 186 | 188 | 189 | 195 | 205 | 205 | 212 |
| | average | 188 | 189 | 189 | 196 | 204 | 204 | 212 |
| | SD | 2.89 | 0.58 | 3.00 | 3.06 | 4.16 | 3.21 | 3.00 |
| Group 4 | 10 | 195 | 193 | 192 | 196 | 200 | 199 | 208 |
| ABI-009 | 11 | 181 | 182 | 189 | 193 | 195 | 198 | 202 |
| 5 mg/kg | 12 | 196 | 197 | 190 | 195 | 204 | 202 | 208 |
| | average | 191 | 191 | 190 | 195 | 200 | 200 | 206 |
| | SD | 8.39 | 7.77 | 1.53 | 1.53 | 4.51 | 2.08 | 3.46 |
| Group 5 | 13 | 182 | 179 | 182 | 183 | 191 | 192 | 198 |
| ABI-009 | 14 | 188 | 180 | 187 | 189 | 193 | 198 | 197 |
| 10 mg/kg | 15 | 190 | 183 | 189 | 193 | 198 | 195 | 204 |
| | average | 187 | 181 | 186 | 188 | 194 | 195 | 200 |
| | SD | 4.16 | 2.08 | 3.61 | 5.03 | 3.61 | 3.00 | 3.79 |

2. Local Toxicity

Fifteen formalin-fixed rat skin samples from the region of SC administration were subject to histopathologic measurement. Histopathologic findings in skin samples included necrosis and mixed infiltrates of inflammatory cells in perivascular zones; both lesions were observed in the subcutaneous tissues/subcutis.

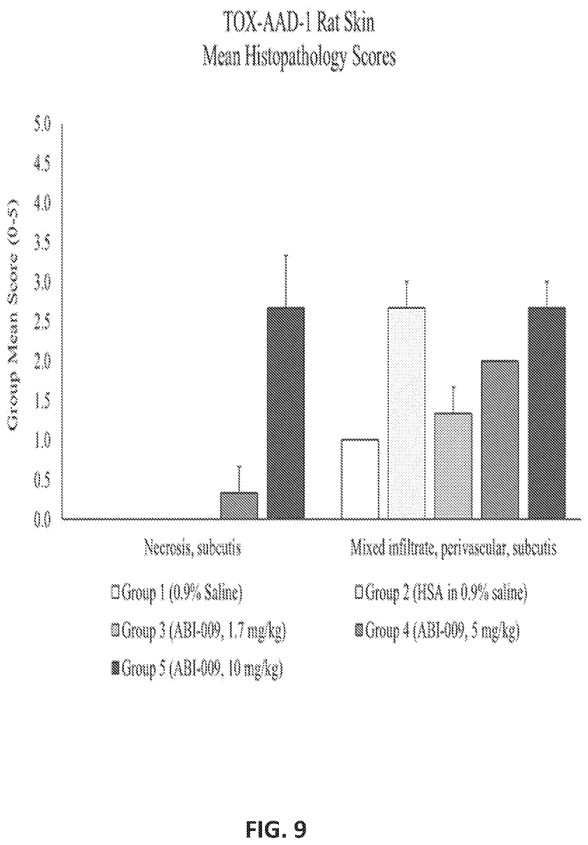

Necrosis was focal and characterized by a region of loss of normal cells, neutrophil infiltration, hemorrhage, and fibrin exudation, with variable adjacent fibroplasia. Necrosis was only observed in samples from animals treated with ABI-009 at 5 mg/kg (Group 4, 1 animal with minimal necrosis) and 10 mg/kg (Group 5, all 3 animals with mild to marked necrosis) dose levels, whereas saline (Group 1), HSA (Group 2), and ABI-009 at 1.7 mg/kg (Group 3) caused no necrosis. See Table 8 and FIG. 9. Only ABI-009 at the highest dose of 10 mg/k-g showed significantly increased necrosis score compared with HSA group (P=0.02, t-test).

TABLE 8
Effect of Treatment on the Body Weight of Rats

| | | | Mixed infiltrate, |
|---|---|---|---|
| | | Necrosis, | perivascular, |
| Group | Sample | subcutis | subcutis |
| Group 1 | 1 | 0 | 1 |
| (0.9% Saline) | 2 | 0 | 1 |
| | 3 | 0 | 1 |
| | mean | 0.00 | 1.00 |
| | SEM | 0.00 | 0.00 |
| Group 2 | 4 | 0 | 2 |
| (HSA in | 5 | 0 | 3 |
| 0.9% saline) | 6 | 0 | 3 |
| | mean | 0.00 | 2.67 |
| | SEM | 0.00 | 0.33 |
| | p vs Grp 1 | | 0.01 |
| Group 3 | 7 | 0 | 1 |
| (ABH-009, | 8 | 0 | 2 |
| 1.7 mg/kg) | 9 | 0 | 1 |
| | mean | 0.00 | 1.33 |
| | SEM | 0.00 | 0.33 |
| | p vs Grp 2 | | 0.05 |
| Group 4 | 10 | 0 | 2 |
| (ABI-009, | 11 | 1 | 2 |
| 5 mg/kg) | 12 | | |
| | mean | 0.33 | 2.00 |
| | SEM | 0.33 | 0.00 |
| | p vs Grp 2 | 0.37 | 0,12 |
| Group 5 | 13 | 2 | 3 |
| (AB1-009, | 14 | 4 | 3 |
| 10 mg/kg) | 15 | 2 | 2 |
| | mean | 2.67 | 2.67 |
| | SEM | 1.00 | 0.00 |
| | p vs Grp 2 | 0.02 | 1.00 |

Mixed inflammatory cell infiltration in subcuticular perivascular zones was characterized by infiltration and aggregation of lymphocytes, plasma cells, macrophages, occasional multinucleated giant cells, and variable numbers of neutrophils. Mixed inflammatory cell infiltration was observed in all treatment groups, with mean scores being the highest in animals treated with HSA (Group 2) and ABI-009 at 10 mg/kg (Group 5). For low dose ABI-009 injection at 1.7 mg/kg (Group 3), the mean score was similar to control group receiving saline injection (Group 1). See Table 8 and FIG. 9. High mixed inflammatory cell infiltration observed in the HSA group (Group 2) compared with saline control (P=0.01, t-test) suggests that local inflammation was largely caused by the injection of the heteroprotein human serum albumin.

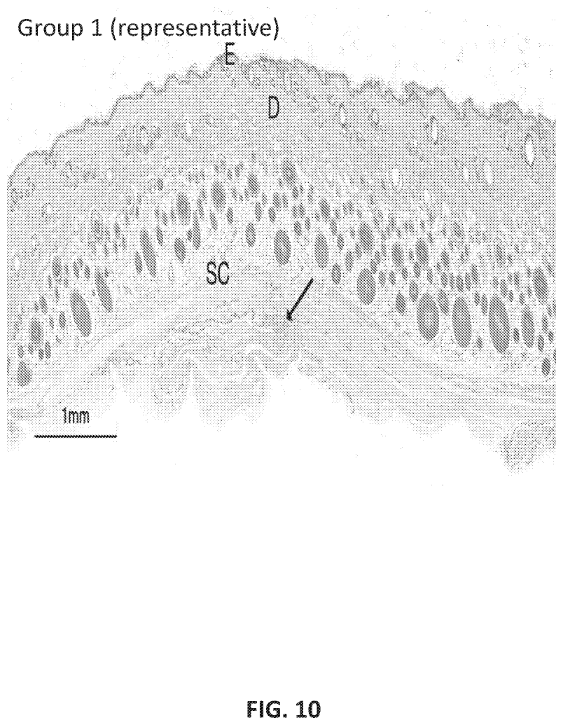

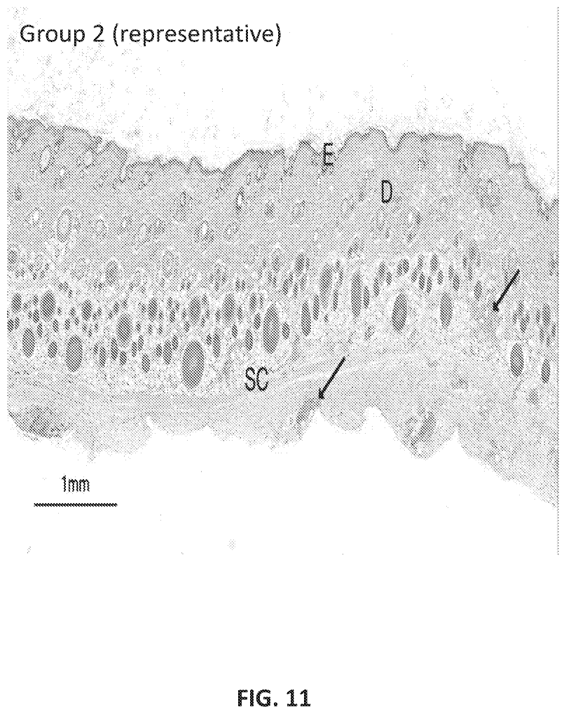

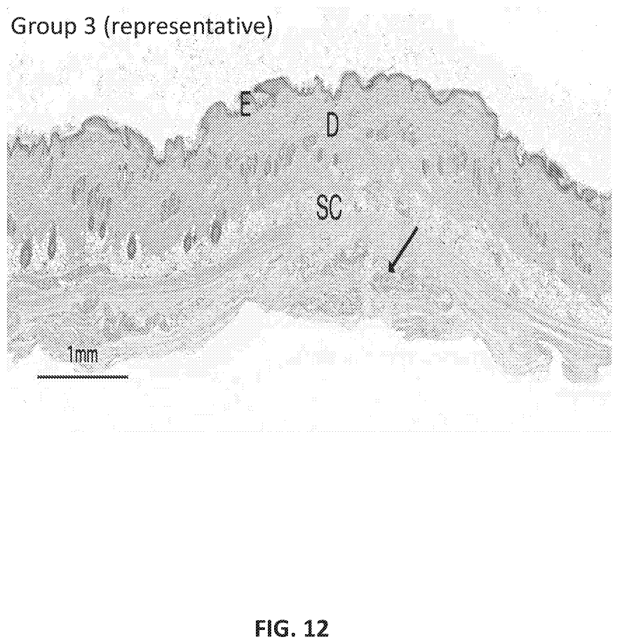

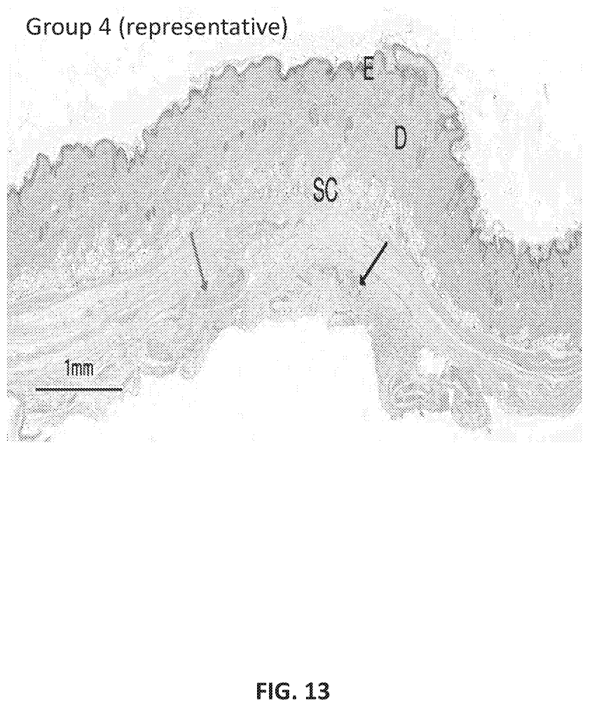

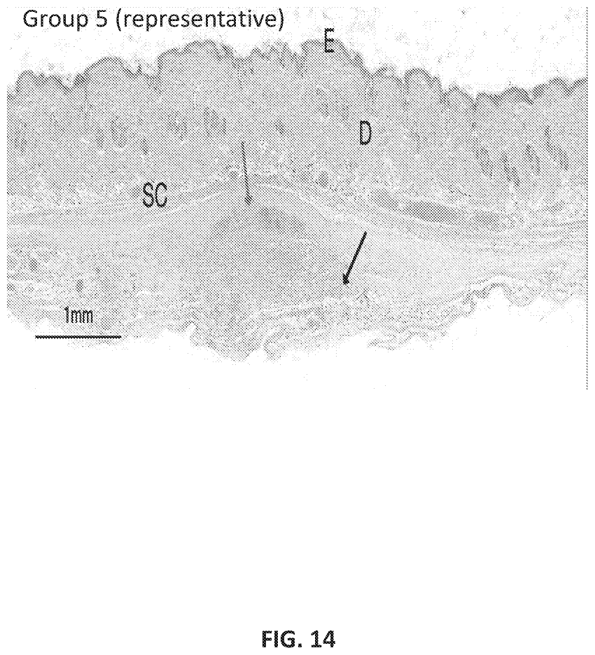

Representative histology images for rats in each group were shown in FIGS. 10-14.

For ABI-009 treatment groups, there were dose-associated increases in local toxicities with increasing ABI-009 dose. At the lowest dose of ABI-009 1.7 mg/kg, the histology of local injection sites was similar to the saline control group; whereas necrosis and subcutaneous tissue inflammatory cell infiltration were the most severe in the ABI-009-treated animals at the 10 mg/kg dose level.

3. Trough Rapamycin Blood Levels

Trough rapamycin blood samples were collected before each injection (at Day 5, 9, 13, 17, 21, 25, 29) for groups treated with ABI-009 (except the 1^{st} dose on Day 1) and analyzed by BASi using LC/MS/MS method. Individual trough levels are shown in Table 9. Most trough rapamycin blood levels 4 days after SC injection were consistently in the range of 2-20 ng/ml. Two samples in the ABI-009 10 mg/kg group (Group 5) were clearly outliers. The reason for this observation cannot be ascertained. However, the abnormal high trough levels only occurred in the highest ABI-009 dose group that also showed mild to marked necrosis in the subcutaneous tissue, suggesting that skin lesions may hamper the normal absorption of ABI-009 and lead to prolonged drug retention.

TABLE 9
Trough Rapamycin Blood Levels

| | Group 3 | | | Group 4 | | | Group 5 | | |
|---|---|---|---|---|---|---|---|---|---|
| Days/ | (ABI-009 1.7 mg/kg) | | | (ABI-009 5 mg/kg) | | | (ABI-009 10 mg/kg) | | |
| ID | #3-7 | #3-8 | #3-9 | #4-10 | #4-11 | #4-12 | #5-13 | #5-14 | #5-15 |
| 5 | 3.1 | 2.38 | 2.56 | 4.5 | 3.63 | 6 | 3.28 | 8.37 | 4.54 |
| 9 | 5.56 | 7.91 | 4.16 | 6.42 | 4.57 | 7.67 | 19.1 | 19.3 | 4.64 |
| 13 | 2.92 | 3.1 | 3.35 | 18.3 | 5.97 | 9.8 | 4.9 | 6.64 | 3.87 |
| 17 | 4.02 | 13 | 2.04 | 1.58 | 3.64 | 9.7 | 11.4 | 6.79 | 14.8 |
| 21 | 0.24 | 1.69 | 3.39 | 3.44 | 3.63 | 4.8 | ALQ | 6.83 | 5.27 |
| | | | | | | | 201* | | |
| 25 | 5.32 | 2.18 | 3.06 | 7.03 | 4.5 | 19.7 | 3.28 | 8.34 | 5.6 |
| 29 | 3.04 | 3.17 | 2.77 | 5.1 | 3.64 | 9.03 | 4.34 | 4.69 | 92.8* |

| | | | |
|---|---|---|---|
| Mean | 3.760 | 6.793 | 7.683 |
| SEM | 0.5736 | 1.005 | 1.139 |

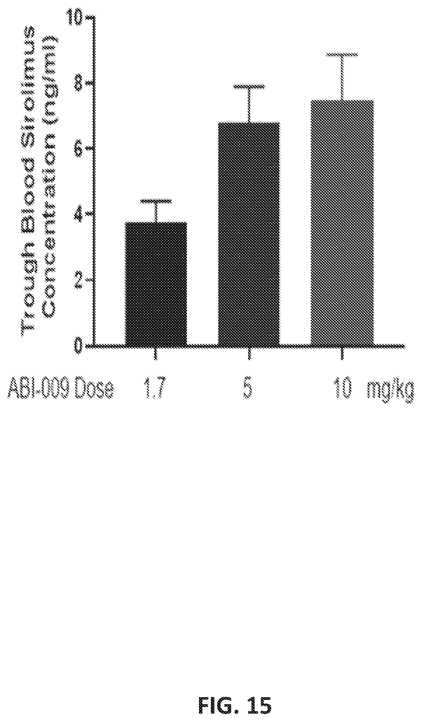

For each ABI-009 treatment group, there was no significant drug accumulation over the time course of the study, as trough blood rapamycin levels remained generally stable. There was a dose-dependent increase in mean trough blood rapamycin levels with increasing ABI-009 dose. Compared with ABI-009 1.7 mg/kg group, higher trough levels were observed in ABI-009 5 mg/kg group (P=0.06) and 10 mg/kg group (P=0.01) (FIG. 15).

In summary, rats were normal post dosing of ABI-009 at current dose regimen (1.7-10 mg/kg, 7 doses), with no body weight loss observed during the study. The histopathology results demonstrated dose-associated local signs of toxicity, with mild to marked necrosis at the highest ABI-009 dose (10 mg/kg). Mixed inflammation cells infiltration may possibly be caused by the heteroprotein HSA. ABI-009 at 1.7 mg/kg (solution concentration 1.7 mg/ml) showed local injection responses similar to saline control. There was no significant drug accumulation following repeated SC injections. Trough blood rapamycin levels increased with higher ABI-009 dose.

The results showed that rats tolerated systemically with multiple doses of ABI-009 over a range of 1.7-10.0 mg/kg with subcutaneous injections. Locally. ABI-009 solution at 1.7 mg/ml concentration was well tolerated. There was no adverse effect observed for this dosage level.

### Example 5: Antitumor Activity Study of Nab-Rapamycin

A study was undertaken to compare the antitumor activity of rapamycin by oral route (Rapamune) and nab-rapamycin (ABI-009) by intravenous or subcutaneous route in a human hepatocellular carcinoma xenograft mouse model.

Human cancer cells were prepared for injection in mice by thawing frozen (by liquid nitrogen) SNU-398 (TSC2-deficient human liver hepatocellular carcinoma cells) obtained from ATCC® (CRL-2233™). Cells were dispersed into a 75 cm² flask containing RPMI 1640 media supplemented with 10% fetal bovine serum and incubated at 37° C. in humidified 5% CO₂. At 80% cell confluence, cells were expanded to 150 cm² flasks with fresh culture media. Cells were grown to obtain a target of 1×10⁷ cells per mouse flank (2×10⁷ per mouse).

20 athymic nude mice were housed in filter-topped cages. Cancer cells were injected subcutaneously into both flanks (1×10⁷ per flank) in 0.1 ml phosphate-buffered saline with 20% Matrigel®.

Treatment Day 1 began with the presence of tumors (tumor average ˜100-150 mm³). Animals were sorted into 4 groups.

Group 1, comprising 5 mice, received saline by intravenous route 2× weekly for 6 weeks.

Group 2, comprising 5 mice, received ABI-009 at 7.5 mg/kg by intravenous route 2× weekly for 6 weeks. Total rapamycin dose was 15 mg/kg/wk.

Group 3, comprising 5 mice, received rapamune at 3 mg/kg 5× weekly for 6 weeks by oral administration. Total rapamycin dose was 15 mg/kg/wk.

Group 4, comprising 3 mice, received ABI-009 at 7.5 mg/kg by subcutaneous route 2× weekly for 6 weeks. Total rapamycin dose was 15 mg/kg/wk.

Measurements (mouse weight and tumor measurements) are made three-times weekly (Monday, Wednesday, and Friday) until predefined sacrifice time points and termination 6 weeks later or when tumors reach maximum volume of 2,000 mm³. Signs of distress will be recorded daily. Tumors will be harvested and stored. Blood samples will be collected at the same time with tumor harvest.

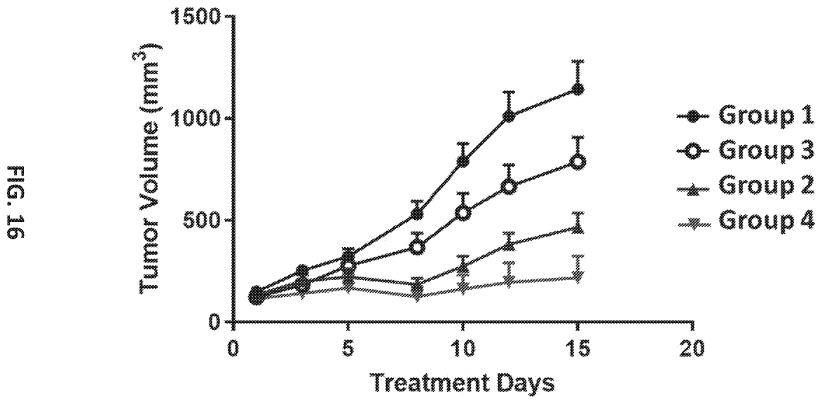

Results: The study is ongoing. Preliminary tumor volume results (mean and standard error of mean, SEM) of each group are summarized in Table 10, below. The tumor growth inhibition (TGI) compared to saline (group 1) and P-value of the TGI vs. saline are reported in Table 10, as well. The results are also summarized in FIG. 16.

TABLE 10
Tumor Growth During Treatment

| Treatment | Group 1 (control) | | Group 2 | | Group 3 | | Group 4 | |
|---|---|---|---|---|---|---|---|---|
| Day | Mean | SEM | Mean | SEM | Mean | SEM | Mean | SEM |
| 1 | 149.2 | 16.8 | 134.6 | 10.9 | 122.6 | 14.5 | 115.9 | 22.3 |
| 3 | 253.6 | 28.3 | 202.0 | 29.7 | 182.9 | 20.0 | 142.0 | 43.6 |
| 5 | 323.5 | 37.0 | 222.4 | 39.7 | 276.7 | 43.2 | 167.6 | 67.2 |
| 8 | 530.6 | 62.9 | 185.9 | 30.2 | 367.9 | 68.6 | 126.2 | 47.9 |
| 10 | 789.4 | 87.8 | 274.5 | 48.4 | 537.4 | 94.6 | 162.8 | 68.8 |
| 12 | 1010.8 | 118.8 | 381.7 | 55.2 | 666.1 | 104.0 | 195.1 | 95.0 |
| 15 | 1142.9 | 136.1 | 465.7 | 68.9 | 786.6 | 120.2 | 217.5 | 106.3 |
| TGI | NA | — | 66.7% | — | 33.2% | — | 89.8% | — |
| P-value vs. | NA | — | 0.0006 | — | NS | — | 0.0001 | — |
| Group 1 | | | | | | | | |

Rapamune oral solution (group 3) at 15 mg/kg/wk resulted in modest tumor growth inhibition (TGI 33.2%, P=not significant) compared with saline control. Equal weekly doses of ABI-009 intravenously (group 2) resulted in significantly greater TGI than oral Rapamune (TGI 66.7% vs saline control, P=0.0016 vs oral Rapamune). However, ABI-009 by subcutaneous route (group 4) produced the most profound tumor growth inhibition (TGI 89.8%, P=0.0001 vs. saline control, P<0.0001 vs oral Rapamune).

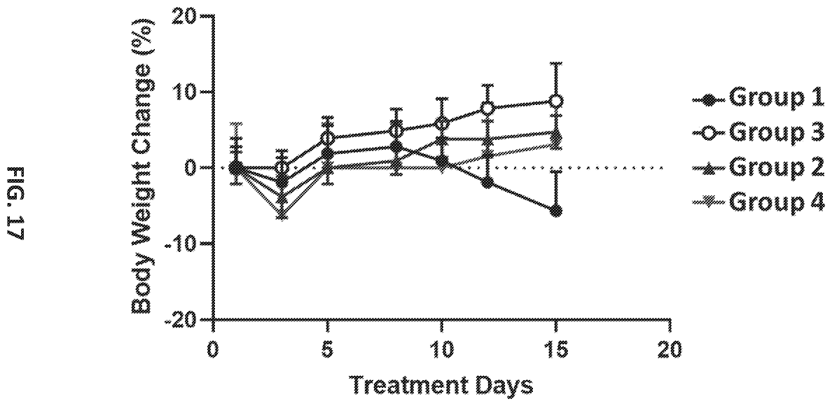

No signs of toxicity were observed in any treatment group. No major weight loss (>10%) were observed in any treatment group. Slight weight loss was observed in the saline control group (group 1) by Day 15, while each treatment group (groups 2-4) maintained body weight or gained weight by Day 15. The body weight results are summarized in FIG. 17.

In conclusion, ABI-009 administered by intravenous or subcutaneous route resulted in significantly greater antitumor activity compared with equal weekly dose of oral Rapamune in a TSC2-deficient SNU-398 human hepatocellular carcinoma xenograft mouse model. ABI-009 by subcutaneous route was surprisingly effective even compared to ABI-009 by intravenous route. No major toxicity or weight loss were observed in any treatment group.

What is claimed is:

1. A method of treating a disease in an individual, comprising subcutaneously administering to the individual a pharmaceutical composition comprising nanoparticles comprising an mTOR inhibitor and an albumin, wherein the amount of the mTOR inhibitor in the pharmaceutical composition is at a dose of about 0.1 mg/m² to about 10 mg/m² for each administration.

2. The method of claim 1, wherein the amount of the mTOR inhibitor in the pharmaceutical composition is at a dose of about 1 mg/m² to about 10 mg/m².

3. The method of claim 1 or claim 2, wherein the amount of the mTOR inhibitor in the pharmaceutical composition is at a dose of about 5 mg/m².

4. The method of any of claims 1-3, wherein the pharmaceutical composition further comprises a saccharide.

5. The method of any of claims 1-4, wherein the pharmaceutical composition is administered once per week or less.

6. The method of claim 5, wherein the pharmaceutical composition is administered once per week.

7. The method of claim 5, wherein the pharmaceutical composition is administered twice every three weeks.

8. The method of any one of claims 1-7, wherein the disease is a cancer.

9. The method of any one of claims 1-7, wherein the disease is a mitochondrial disease.

10. The method of any one of claims 1-9, wherein the individual is a human.

11. A method of delivering an effective amount of an mTOR inhibitor to a target tissue of an individual, the method comprising subcutaneously administering a pharmaceutical composition comprising nanoparticles comprising an mTOR inhibitor and an albumin.

12. The method of claim 11, wherein the mTOR inhibitor is a limus drug.

13. The method of claim 12, wherein the limus drug is rapamycin.

14. The method of any one of claims 11-13, wherein the pharmaceutical composition further comprises a saccharide.

15. The method of any one of claims 11-14, wherein the amount of the mTOR inhibitor in the pharmaceutical composition is at a dose of about 0.1 mg/m² to 10 mg/m².

16. The method of any one of claims 11-15, wherein the target tissue is a brain tissue of the individual.

17. A pharmaceutical composition suitable for subcutaneous administration to an individual comprising: a) nanoparticles comprising an mTOR inhibitor and an albumin, and b) a saccharide.

18. The pharmaceutical composition of claim 17, wherein the saccharide is selected from the group consisting of alginate, a starch, lactose, pullulan, hyaluronic acid, chitosan, glucose, galactose, mannose, N-acetylglucosamine, sucrose, N-acetyl-D-galactosamine, maltose, or trehalose.

19. The pharmaceutical composition of claim 18, wherein the saccharide is sucrose.

20. The pharmaceutical composition of claim 18, wherein the saccharide is trehalose.

21. The pharmaceutical composition of any one of claims 17-20, wherein the concentration of mTOR inhibitor in the pharmaceutical composition is at least about 5 mg/ml.

22. The pharmaceutical composition of any one of claims 17-21, wherein the concentration of mTOR inhibitor in the pharmaceutical composition is at least about 50 mg/ml.

23. The method of any one of claims 1-16 or the pharmaceutical composition of any one of claims 17-22, wherein the average diameter of the nanoparticles in the pharmaceutical composition is no greater than about 120 nm.

24. The method of any one of claims 1-16 or the pharmaceutical composition of any one of claims 17-23, wherein the nanoparticles comprise the mTOR inhibitor coated with the albumin.

25. The method of any one of claims 1-16 or the pharmaceutical composition of any one of claims 17-24, wherein the albumin is human albumin.

26. The method of any one of claims 1-16 or the pharmaceutical composition of any one of claims 17-25, wherein the mTOR inhibitor is a limus drug.

27. The method or the pharmaceutical composition of claim 26, wherein the mTOR inhibitor is rapamycin.

28. A device for subcutaneously administering to an individual a pharmaceutical composition comprising nanoparticles comprising an mTOR inhibitor and an albumin, the device comprising:
a) a drug chamber containing a dried form of the pharmaceutical composition, and a solution chamber containing a reconstituting solution:
b) a removable divider separating the drug chamber and the solution chamber, wherein removal of the divider causes mixing of the dried pharmaceutical composition and reconstituting solution, thereby forming a reconstituted pharmaceutical composition.

29. The device of claim 28, wherein the device is a syringe, the syringe comprising an injection needle affixed to an end of the syringe and a pusher capable of expelling the reconstituted pharmaceutical composition from the syringe.

30. The device of claim 28 or claim 29, wherein the pharmaceutical composition further comprises a saccharide.

31. The device of any one of claims 28-30, wherein the mTOR inhibitor is a limus drug.

32. The device of any one of claims 28-31, wherein the mTOR inhibitor is rapamycin.

33. A kit comprising the device of any one of claims 28-32 for use in treating a disease.

34. The kit of claim 33, further comprising instructions for using the kit to treat cancer or a mitochondrial disease.
